(19) United States
(12) Patent Application Publication
(10) Pub. No.: US 2011/0104296 A1
(43) Pub. Date: May 5, 2011

(54) Endorphin Therapy Compositions and Methods

(76) Inventor: Dipak Kumar Sarkar, Skillman, NJ (US)

(21) Appl. No.: 12/990,896

(22) PCT Filed: May 8, 2009
(86) PCT No.: PCT/US09/02894

§ 371 Date: Dec. 22, 2010

Related U.S. Application Data
(60) Provisional application No. 61/051,668, filed on May 8, 2008.

(51) Int. Cl. A61K 9/00; C12N 5/0797; C12N 5/0793; A61K 38/43; A61K 38/51; A61P 35/00; A61P 35/04; A61P 37/04; A61P 31/12; A61P 31/04; A61P 3/10; A61P 19/02; A61P 5/14; A61P 29/00; A61K 38/54; B82Y 5/00

(52) U.S. Cl. 424/499; 435/378; 435/375; 435/377; 424/94.1; 424/94.5; 424/204.1; 424/234.1; 424/277.1; 424/94.2; 977/795; 977/915

(57) ABSTRACT

In certain embodiments, the invention provides methods of isolating and culturing neuronal stem cells from hypothalmi, methods of differentiating the neuronal cells into beta-endorphin neurons, and methods of treatment of various diseases comprising administering agents to differentiate endogenous neuronal stem cells into beta endorphin neurons.

### CROSS-REFERENCE TO RELATED APPLICATIONS

This application claims priority to U.S. Provisional Patent Application No. 61/051,668 filed on May 8, 2008, the disclosure of which is hereby incorporated by reference in its entirety.

### FIELD OF THE INVENTION

The present invention relates to compositions and methods to increase the body's innate immunity to prevent tumor cell growth and immune-related diseases. More specifically, the present invention relates to beta-endorphin compositions and methods to increase innate immunity.

### SUMMARY OF THE INVENTION

A novel method to isolate neural stem cells (NSC) from fetal hypothalamus has been developed. The methods include preparation of dissociated cells from fetal rat hypothalami. Then, purifying these mixed neuronal, glial and stem cells from mixed neural and neural progenitor cells by the use of uridine and fluodeoxyuridine to kill the glial cells and leaving the live neural and neural stem cells in cultures. After selection, the method comprises growing these cells for several generations in cultures so that only the neural stem cells remain in cultures. Once this is achieved, the cells are maintained in cultures in the presence of stem cell medium with lymphokine inhibiting factor (LIF; about 0.1 micro gram/ml) and basic fibroblast growth factor (bFGF; about 20 ng/ml) so that only neural stem cells with the ability to differentiate into beta-endorphin (BEP) neurons remain in cultures.

A method to differentiate beta-endorphin neuronal cells from neural stem cells has also been developed. Neural stem cells can be differentiated to beta-endorphin neurons if they were removed from the influence of LIF and then maintaining them in the environment favoring the survival of neurons (e.g., neuron culture media) and then treating them for about 1 week with pituitary adenylate cyclase activating peptide (PACAP) and dibutyryl cyclic adenylate cyclase (dbcAMP). Beta-endorphin neuron purity increases when neural stem cells were treated with both of these agents at about 10 micromolar dose/each but other doses are also effective at different efficiency. Treatment with only dbcAMP or PACAP is also effective but with less efficiency. Application of dbcAMP or cAMP activating agent is also effective to differentiate endogenous neural stem cells if these agents are delivered to the brain via a delivery system like nanospheres or other vehicles.

It has been discovered that beta-endorphin cells inhibit body stress responses, activate natural killer (NK) cells and inhibit proinflammatory cytokines.

NK cells are mediators of the innate immune response critical for defense against infectious viral and bacterial diseases (e.g. AIDS, etc.) and cancers (e.g., prostate, breast, etc.). Increases in innate immunity by beta-endorphin cells may provide a unique approach to combat cancer and cancer metastasis, various immune diseases, and pathogenic infections. Reduction of inflammatory cytokines not only prevents tumor growth and progression but also reduces other diseases associated with inflammations such as rheumatoid arthritis development. Additionally, these cells suppress stress axis function and thereby are beneficial in stress reduction and in controlling stress-induced metabolic diseases.

Market Applications: Therapeutics, Cancers (prostate, breast and other cancers), Tumor Metastasis, Infectious Diseases, Stress Control, Metabolic Diseases, Fetal alcohol patients stress and immune related problems

Advantages: Studies show the ability of the in vitro produced beta-endorphin cells maintain functionality in vivo and increase NK cytolytic activity. Initial pre-clinical models of prostate and breast cancers demonstrate the potential for enhancing innate immunity to prevent cancer growth and progression and metastatic invasion. In preclinical model of stress control identify a significant beneficial effect of beta-endorphin cell therapy in stress reduction in fetal alcohol exposed subjects. Furthermore, beta-endorphin cells showed unique ability to reduce the incidence of rheumatoid arthritis.

In certain embodiments, the invention is directed to a method of isolating neural stem cells from a fetal hypothalamus comprising: isolating mixed neural and neural stem cells from glial cells in a fetal hypothalamus, and growing the isolated mixed neural and neural stem cells for several generations in cultures so that only the neural stem cells remain in the cultures.

In certain other embodiments, the method of further comprises introducing an agent that kills the glial cells but not the neural and neural stem cells in cultures.

In certain other embodiments, the agent is selected from the group consisting of uridine, fluodeoxyuridine and a combination thereof.

In accordance with any of the above embodiments, the invention further comprises maintaining the neural stem cells in cultures in the presence of stem cell medium with lymphokine inhibiting factor (LIF) so that only neural stem cells with the ability to differentiate into beta-endorphin (BEP) neurons remain in cultures.

In certain other embodiments, the concentration of LIF is about 0.1 microgram/ml.

In certain other embodiments, the cultures also contain basic fibroblast growth factor (bFGF).

In certain other embodiments, the concentration of bFGF is about 20 ng/ml.

In accordance with any of the above embodiments, the invention is further directed to a method of differentiating beta-endorphin neuronal cells from neural stem cells that are under the influence of LIF comprising: (i) removing the influence of LIF from the neural stem cells, (ii) maintaining the neural stem cells in an environment favoring the survival of neurons, and then (iii) treating the neural stem cells with a differentiating agent selected from group consisting of (a) pituitary adenylate cyclase activating peptide (PACAP), (b) dibutyryl cyclic adenylate cyclase (dbcAMP) and (c) a combination thereof.

In certain embodiments, the environment in step (ii) is neuron culture media.

In certain other embodiments, the treating in step (iii) is performed for about 7 days.

In certain other embodiments, the agent in step (iii) is a combination of PACAP and dbcAMP.

In still other embodiments, the dose of the agent is about a 10 micromolar dose.

In certain other embodiments, the dose of PACAP is about a 10 micromolar dose and the dose of dbcAMP is about a 10 micromolar dose.

The invention is also directed to a method of differentiating endogenous neural stem cells into BEP cells in a patient in need thereof comprising: (i) administering an effective amount of an agent selected from the group consisting of (a) pituitary adenylate cyclase activating peptide (PACAP), (b) dibutyryl cyclic adenylate cyclase (dbcAMP) and (c) a combination thereof into the central nervous system.

In certain embodiments, the agent is administered into the brain.

In certain other embodiments, the agent is administered into the hypothalamus.

In certain other embodiments, the agent is administered into the third ventral.

In accordance with any of the above embodiments, the invention is also directed to a method wherein the agent is administered as a pharmaceutically acceptable nanosphere.

In accordance with any of the above embodiments, the invention also provides a method wherein the amount of agent administered is sufficient to provide BEP cell differentiation to reduce physiological stress responses.

In certain other embodiments, the amount of agent administered is sufficient to provide BEP differentiation to activate natural killer (NK) cells and inhibit proinflammatory cytokines.

In certain other embodiments, the amount of agent administered is sufficient to improve the innate immune response critical for defense against diseases selected from the group consisting of infectious diseases including viral and bacterial diseases, and hyperproliferative diseases such as cancers.

In accordance with any of the above embodiments, the invention is also directed to a method wherein the disease is neoplasia.

In certain other embodiments, the disease is prostate cancer.

In certain other embodiments, the disease is breast cancer. In still other embodiments, the disease is metastatic breast cancer.

In certain other embodiments, the amount of agent is administered is sufficient to provide BEP cell differentiation to reduce inflammation associated with immunologic diseases.

In certain other embodiments, the immunological disease is selected from the group consisting of rheumatoid arthritis, adult onset diabetes (type II), obesity, thyroid disorder, celiac disease, inflammatory bowel syndrome, lupus and a combination thereof.

### BRIEF DESCRIPTION OF THE DRAWINGS

FIG. 1 is an image of neuronal stem cells. Characterization of hypothalamic neuronal stem cells in cultures. (A-C) Phase-contrast images of embryonic rat hypothalamic neuronal stem cells at the stage of aggregated primary sphere (A) and single (B) and aggregated (C) secondary spheres. (D) Immunofluorescence staining (green color) of nestin for primary single spheres (small arrows) or aggregated spheres (large arrows). Secondary spheres also stained for nestin (E) or vimentin (F). “−”=20 μm.

FIG. 2 is an image of neuronal stem cells. Characterization of hypothalamic neuronal stem cells at various phases of differentiation by PACAP and cAMP in cultures. (A) Phase-contrast images of neuronal stem cells treated with 10 μM of PACAP and 10 μM of cAMP for a period of 3 d. (B, C) Representative photograph showing the immunofluorescence staining for vimentin (B) and ct-internexin (C) in the early phase of differentiation at 3 d. “−”=20 μm. (D) Phase-contrast images of neuronal stem cells treated with 10 μM of PACAP and 10 μM of cAMP for a period of 1 week. (E) A representative photograph showing the immunofluorescence staining for the neuronal marker NF-M (shown in green). (F) Immunofluorescence staining for BEP shown in red. (G) Phase-contrast images of control neuronal stem cells maintained without the PACAP and cAMP in neuronal culture medium. (H) No staining was seen when these control undifferentiated neuronal stem cells were stained for BEP. “−”=20 μm.

FIG. 3 is an image of neuronal stem cells. Characterization of PACAP- and cAMP-induced differentiated hypothalamic neuronal stem cells in cultures. (A) Phase-contrast images of neuronal stem cells treated with 10 μM of PACAP and 10 μM of cAMP for a period of 1 week and then maintained in serum-free defined neuron culture media without the differentiation factors for a period of 1 week. (B & C) A representative photograph showing the immunofluorescence staining (shown in green) for neuronal markers MAP2 (B) and type III β-tubulin (C). (D) No staining was seen when these cells were stained for a glial marker GFAP. (E) Immunofluorescence staining for BEP shown in red. (F) BEP staining was absent when cells were stained with the BEP antibody that was preincubated with excess antigen. The blue staining in this figure represent the DAPI staining for the nucleus of the cells in culture. “−”=20 μm.

FIG. 4 is an image of neuronal stem cells. Characterization of neuropeptide production by the PACAP- and cAMP-induced differentiated hypothalamic neuronal stem cells in cultures. Representative photographs showing the immunofluorescence staining (shown in red) for various cAMP-responsive neuropeptides (□-endorphin, BEP; neuropeptide y, NPY; gonadotropin releasing hormone, GnRH; tyrosine hydroxylase, TH). The blue staining represents DAPI staining for the nucleus of the cells. Note that the differentiated NSC gave the appearance of violet color because of the combined red color for BEP and blue color for cell nucleus. “−”=20 μm.

FIG. 5 is a graphical depiction showing characterization of the basal and PgE1-induced increase in BEP release and POMC mRNA expression from hypothalamic neuronal stem cells after PACAP- and cAMP-induced differentiation. (A & B) Shows the time characteristic of the BEP release (A) and POMC mRNA expression (B) from neuronal stem cells differentiated in the presence of PACAP (10 μM) and dbcAMP (10 μM). No BEP was detected in media samples collected from control cultures without the PACAP and dbcAMP treatment (0-day) or the cultures treated with the differentiating agents for 3-day. P<0.05, significantly different from the values of the rest of the groups. (C & D) Demonstrates the dose-response and synergistic effects of PACAP and dbcAMP on BEP release (C) and POMC mRNA expression (D) from differentiated neuronal stem cells treated with the drugs for 1 week and then without the drugs for 1 week. The control group was treated similarly with vehicle. P<0.05, significantly different from the values of the 1-μM dose of the similar agent. P<0.05, significantly different from the values of the rest of the groups. (E & F) Shows the PgE1 (10 μM)-induced BEP release response (E) and POMC mRNA expression response (F) from differentiated neuronal stem cells treated as in FIG. 5a. Values are presented as a percentage of vehicle-treated control. P<0.05, significantly different from the values of control. P<0.05, significantly different from the values at 1 week.

FIG. 6 A-D is an image of neuronal stem cells. Determination of in vivo functionality of the PACAP and dbcAMP-induced differentiated hypothalamic neuronal stem cells. (A-D) Immunocytochemical characterization of differentiated neuronal stem cells in the presence of PACAP (10 μM) and dbcAMP (10 μM) at 2 weeks after transplantation into the PVN of the hypothalamus in male rats. Representative photographs showing BrdU-stained cells at the site of transplantation (A). “−”=10 μM. High-power view of a photograph showing immunofluorescence staining for BrdU (green) and BEP (red) of transplanted cells (B). Many BEP-stained cells show long processes (arrows; C) “−”=20 μM. Merged images from B and C show numerous BEP- and BrdU-double stained cells (D). FIG. 6 E-G is a graphical depiction showing in vivo functionality of the PACAP and dbcAMP-induced differentiated hypothalamic neuronal stem cells. (E) POMC mRNA levels in the lobe of PVN with transplanted differentiated neuronal stem cells (TP) and in the contralateral lobe of the PVN that underwent sham-transplant surgery (S-TP). P<0.05, significantly different from the values of S-TP. (F-G) Physiological responses of transplanted cells. POMC mRNA (F) and CRH mRNA (G) levels in the PVN lobe with TP and in the contralateral lobe of the PVN with S-TP after i.p. administration of LPS or of saline in pubertal male rats exposed to alcohol in fetal life (FAE). The effect of LPS or saline treatment in pubertal male rats exposed to no alcohol in fetal life (control) was shown for comparison. N=6-8. ^{a}, P<0.05, vs. saline. P<0.05, vs. LPS-treated control or S-TP animals.

FIG. 7 is a graphical depiction of a determination of the effect of NSC-BEP transplants on NK cell cytolytic function. Adult male rats (90 days old) fed during embryonic days 11 through 21 via dams; with alcohol (alcohol-fed rats), an isocaloric liquid diet (pair-fed rats) or with a regular diet (ad lib-fed rats) as described (Arjona et al., 2006) were transplanted with NSC-BEP (BEP; 20,000 cells/1 μl) or cortical cells (CORT; 20,000 cells/1 μl) into the left PVN. After 3 weeks rats were sacrificed and the spleens were collected. Splenocytes were prepared and assayed for NK cell cytolytic activity against YAC-lymphoma cells by a standard 4 hr chromium-51 release cytolytic assay (Boyadjieva et al., 2001). The histograms represent mean±SEM of NK activity in lytic units (LU). N=5-7 rats. P<0.05, vs. ad lib and pair-fed rats. P<0.05, vs. CORT cell-treated or untreated animals fed similarly during embryonic life.

FIG. 8 is a graphical depiction of a determination of the effect of NSC-BEP transplants on NK cell functions: Dose-dependent effect. Adult male rats (90 days old) alcohol-fed, pair-fed or ad lib-fed during the prenatal period were transplanted with NSC-BEP or cortical cells into one PVN (×1) or two PVN (×2). After 3 weeks, approximately 1 ml of blood was collected from the orbital sinus of each rat and used for PBMC preparation and plasma separation. PBMC samples were used to determine the NK cytolytic activity by a standard 4 hr chromium-⁵¹ release cytolytic assay. Plasma samples were used to determine IFN-gamma and TNF-alpha levels by ELISA. (Due to low blood volumes in some samples, TNF-alpha could not be measured in these samples.) The histograms represent meant SEM values from 5-7 rats. P<0.05, vs. CORT-cell transplant in rats that were similarly treated prenatally. P<0.05, vs. BEP×1-cell transplant in rats that were similarly treated prenatally. P<0.05, compared to the rest of the groups.

FIG. 9 is a graphical depiction of a determination that NSC-BEP cell transplants in the paraventricular nuclei of the hypothalamus reduce the ability of carcinogen and hormone to induce prostate tumors. Treatments with NMU and testosterone increased the weight of prostate (a crude measure of tumor in prostate) about 3-fold in pair-fed and ad lib fed rats transplanted with control cells (cortical cells were used as control cells in pair-fed rats, and neuronal stem cells without differentiation were used as control cells in ad lib fed rats; CONT-TP) in the PVNs. Transplantation of NSC-BEP in both PVNs (NSC-BEP-TP) suppressed the ability of carcinogen and hormone to increase prostate weight. P<0.01, significantly different from the control treatments within the similarly-fed group.

FIG. 10 is an image of histopathology of prostate of rats transplanted with control cortical cells, nondifferentiated NSC cells or NSC-BEP cells. The treatment of NMU and testosterone induced significant neoplasia in prostates of rats transplanted with control cortical (top row; showing hyperplasia and adenocarcinoma) or NSC cells (bottom left; showing adenocarcinoma). Prostate histology appears to be mostly normal in NSC-BEP transplanted rats (a representative picture is shown on the bottom right). Magnifications are in 5× except the bottom right, which is in 2×.

FIG. 11 is an image of cancer prostate tissue in evaluation of the effect of BEP cell transplants on the MNU and testosterone-induced prostate cancers. Adult male rats were transplanted with in vitro differentiated BEP cells or cortical cells (CONT) bilaterally in the PVN of male rats. These rats were then treated with MNU and testosterone treatments and used for determination of histopathology of prostates. (A-C) Prostates of rats transplanted with CONT showed lesions ranging from epithelial hyperplasia with mild atypia (A) to high-grade PIN (B) and occasionally invasive adenocarcinoma (shown by arrows; C). (D-E) Prostates of rats transplanted with BEP cells demonstrated very mild changes, ranging from minimal (D) to moderate hyperplasia (shown by arrows; E). “−”=10 μm.

### DETAILED DESCRIPTION OF THE INVENTION

The present invention is described more fully by way of the following. All references cited are incorporated by reference herein.

Abbreviations: ANOVA, analysis of variance; beta-endorphin, BEP; cAMP, cyclic adenosine monophosphate; dbcAMP, dibutyryl cAMP; DMEM, Dulbecco's modified Eagle's medium; DAPI, 4′-6-Diamidino-2-phenylindole; EDTA, ethylenediaminetetraacetic acid; EGF, epidermal growth factor; FBS, fetal bovine serum; FGF, fibroblast growth factor; GABA, gamma-aminobutyric acid; GFAP, glial fibrillary acidic protein; GnRH, gonadotropin hormone-releasing hormone; HDMEM, HEPES-buffered Dulbecco's Modified Eagle's Medium; IgG, immunoglobulin G; kDa, kilodalton; LIF, limphokine inhibitory factor; MAA, Mem amino acid; MAP2, microtuble-associated protein 2; NF-M, Neurofilament M; NPY, neuropeptide Y; NSC, neuronal stem cell; PACAP, pituitary adenylate cyclase-activating polypeptide; POMC, proopiomelanocortin; PGE1, prostaglandin E1; RT-PCR, reverse transcription-polymerase chain reaction, TH, tyrosine hydroxylase.

Cancer immunotherapy is a growing field that aims at restoring and enhancing immune function to combat oncogenic conditions. NK cells play an important role in preventing cancer growth and metastasis, and expansion of these cells in vivo could be a promising immunotherapeutic strategy against cancer either alone or in combination with conventional therapies. However, the use of autologous natural killer (NK) cells is limited due to the fact that selective NK expansion is difficult to achieve in human patients. Opioid peptides can activate NK cell functions in both laboratory animals and in humans. Whether beta-endorphin (BEP) cell therapy may have a significant impact on activating NK cell function to clear cancers cells has not been tested. There was no method to prepare viable primary BEP neurons from the hypothalamus, so it was not feasible to conduct a primary cell replacement therapy study for determining the biological responses. Hence, we determined the feasibility of differentiating neuronal stem cells (NSCs) into BEP neurons in vitro. To begin to examine the capacity of NSCs to generate BEP neurons, we purified neurons from embryonic hypothalamic tissues and grew neurospheres in culture using a stem cell-maintaining medium. These neurospheres were maintained in culture for a period of 2 weeks in the presence or absence of added factors including basic fibroblast growth factor (bFGF). Upon dissociation, they developed secondary neurospheres and formed aggregates that expressed nestin and vimentin, protein markers of the immature uncommitted phenotype. The neurosphere can be maintained in culture for several months by regularly changing medium and splitting cells. The neurospheres were considered NSCs. Pituitary adenylate cyclase-activating peptide (PACAP), a cyclic adenosine monophosphate (cAMP)-activating agent, is highly expressed in the hypothalamus during the period when many neuroendocrine neurons differentiate from the neuronal stem cells (NSCs). We tested the effect of dibutyryl cAMP (dbcAMP) in combination with the PACAP on the differentiation of rat fetal NSCs in primary cultures. The differentiated cells produced neuroendocrine protein BEP but not gonadotropin hormone-releasing hormone (GnRH), neuropeptide Y (NPY) and tyrosine hydroxylase (TH). These cells expressed the BEP peptide-producing gene proopiomelanocortin, and produced an increased amount of the gene and the peptide in response to a regulatory hormone, prostaglandin E. These results suggest that cAMP-elevating agents are involved in differentiation of NSC to BEP neuron. When these cells were transplanted into the paraventricular nuclei (PVN) of the hypothalamus, they maintained functionality for a prolonged period of time, as they show BEP immunoreactivity, POMC gene production and the ability to reduce corticotropin releasing hormone (CRH) gene expression following administration of lipopolysaccaride (LPS). We also investigated whether NSC-BEP cell transplants for a period of 3 weeks activate NK cell function. We studied this by transplanting NSC-BEP cells or control cells in immune-deficient neonatally alcohol-fed male rats during the adult period in one PVN or both PVNs in alcohol-fed and control-fed rats. Cortical cells were used as a control for NSC-BEP. Determination of NK cell functions revealed that NSC-BEP transplants significantly increased NK cell cytolytic activity both in the spleens and in peripheral blood mononuclear cells (PBMC) and IFN-gamma levels in plasma in control-fed and alcohol-fed rats. The activation of NK cytolytic function and plasma levels of IFN-gamma, by NSC-BEP cells were higher when transplanted in both PVNs as compared to only one PVN. Bilateral NSC-BEP cell transplants in both PVNs were also able to increase NK cytolytic function and plasma levels of IFN-gamma in alcohol-fed animals. However, NSC-BEP cell transplants decreased TNF-alpha levels in the plasma of alcohol-fed and control-fed animals. The levels of both basal and LPS-induced NK cytolytic function and IFN-gamma mRNA levels in splenic tissues of alcohol-fed animals transplanted with NSC-BEP neurons in the PVN were higher than levels in alcohol-fed animals with sham transplants in the PVN. These results suggest that NSC-BEP cell transplants are effective in activating NK cell functions. Because NK cells are one of the cellular mediators of innate defense and are crucial for defense against infectious diseases and cancer, it is suspected that NSC-BEP cell transplants may alter tumor cell growth. We determined the effects of the cell transplants on carcinogen (N-nitroso-N-methylurea; NMU) and hormone (testosterone)-induced prostate tumor growth. In this experiment control fed male rats were transplanted with NSC-BEP cells, cortical cells or NSC cells in both PVN and then were treated with carcinogen and hormones. The wet weights of total prostate/kg of body weight were 3-5-fold higher in hormone and carcinogen-treated animals than the untreated control animals. The wet weights of total prostate were 3-4-fold lower in animals treated with NSC-BEP cells than those treated with cortical cells or NSC cells. Limited histopathological data revealed that the prostate of animals treated with control cell transplants have either adenoma or adenocarcinoma but the prostate of animal treated with NSC-BEP cells showed free of adenoma or adenocarcinoma. These data indicate that NSC-BEP cell therapy has the potential to increase the body's innate immunity to prevent cancer growth and progression.

Cancer immunotherapy is a growing field that aims at restoring and enhancing immune function to combat oncogenic conditions. One target of this field is the NK cell. As part of the innate immune system, NK cells form the first line of defense against pathogens or transformed/cancerous host cells. In addition, NK cells are likely to interact with potent antigen-presenting dendritic cells, thus forming a bridge between innate and adaptive immunity. Recent experimental and clinical data show the possibility of exploiting NK activity as a cell-based immunotherapy to treat cancer (reviewed in Arai and Kingemann, 2005). Results from stem cell transplants containing alloreactive donor NK cells and in vitro work indicate a great antitumor potential of NK cells (reviewed in Chaudhuri and Law, 2005).

The natural killer cell is a critical component of the innate immune system and plays a central role in host defense against tumor and virus-infected cells. The importance of the NK cell in controlling tumor growth and metastasis of breast cancer cells has been clearly demonstrated in severe combined immunodeficiency (SCID) mice. It has been shown that breast cancer cells, following inoculation, were efficient in forming large tumors and spontaneous organ-metastasis in NOD/SCID/gammac (null) (NOG) mice lacking T, B and NK cells. In contrast, breast cancer cells produced a small tumor at the inoculated site and completely failed to metastasize into various organs in T and B cell knockout NOD/SCID mice with NK cells. Immunosuppression of NOD/SCID mice by treatment with an anti-maurine TM-beta1 antibody, which transiently abrogates NK cell activity in vivo, resulted in enhanced tumor formation and organ-metastasis in comparison with non-treated NOD/SCID mice. Activated NK cells inhibited tumor growth in vivo. These results suggest that NK cells play an important role in cancer growth and metastasis and could be a promising immunotherapeutic strategy against cancer, either alone, or in combination with conventional therapy (Dewan et al., 2005, 2006).

NK cells are one of the cellular mediators of innate defense. They can recognize and kill aberrant cells and rapidly produce soluble factors (chemokines and cytokines) that have antimicrobial effects or that prime other cells of the immune system (Janeway and Medzhitov, 2002). Heterogeneous arsenal of surface receptors that allow NK cells to respond to microbial products, cytokines, stress signals and inducible molecules are expressed after target-cell transformation (Colucci et al., 2003). NK cells are therefore crucial for defense against infectious diseases and cancer. They play a vital role in cellular resistance to malignancy and tumor metastasis (Miller, 2001; Colucci et al., 2003). NK cells can destroy infected and malignant cells by calcium-dependent release of cytolytic granules, by activation of the Fas (CD95)-mediated pathway, or by tumor necrosis factor-alpha (TNF-α) release (Austin Taylor et al., 2000) and activating TNF-α-related apoptosis-inducing ligand (TRAIL)-dependent receptors (Smith et al., 2002). Among these mechanisms, the release of cytolytic granules containing granzymes (particularly granzyme B) and perforin is the major mechanism used for killing the target cell (Barry and Bleackley, 2002; Raja et al., 2002). Perforin creates transmembrane pores in the target cell membrane thereby allowing the entry of granzyme B, which then activates the caspase-driven apoptotic pathway. NK cells differ from other cytotoxic effector cell types (e.g., cytotoxic T lymphocytes) in two major ways. They kill the target cells in a non-major histocompatibility complex (MHC)-restricted fashion without the need for previous in vitro or in vivo activation, and only NK cells constitutively express the lytic machinery (Trinchieri, 1989; Moretta et al., 2002). In addition, NK cells can perform antibody-dependent cell-mediated cytotoxicity (ADCC), which involves the lysis of antibody-coated targets (Perussia et al., 1983). Another essential function of NK cells is the production of cytokines such as interferon-gamma (IFN-γ), (TNF-α), and granular macrophage cell-stimulating factor (Trinchieri, 1989). NK cells are highly efficient in the cellular immune response against malignant tumors without restriction of major histocompatibility complex. However, clinical studies using autologous NK cells have been reported in only a very limited number of cases, due to the fact that selective NK expansion is difficult to achieve in this patient population (Ishikawa et al., 2004).

One of the endogenous peptides that control NK cell function is beta-endorphin (BEP). Cells producing this peptide are localized in the hypothalamus. These neurons are shown to be important regulators of NK cell activity (Boyadjieva et al., 2001, 2002, 2004; Dokur et al., 2004, 2005). Opioid peptides can affect the NK cell functions by several different mechanisms. It has been shown that endogenous opioids up-regulate human and rat NK cell activity (Faith et al, 1984; Kay et al, 1984, Matthews et al, 1983). The cytolytic activity of NK cells can be enhanced by lymphokines such as IFN-gamma and interleukin-2 (IL-2) (Ortaldo et al, 1983, Santoli et al, 1987). The effects of IFN-gamma on NK cells can be blocked by the opioid antagonist naloxone (Kay et al, 1984). In addition, IL-2 and IFN-gamma have been shown to bind to opioid receptors (Ahmed et al, 1984). Comparison of the effects of IFN-gamma and BEP revealed that NK activity is enhanced by these two agents to the same magnitude (Dafny, 1983). In addition, IFN-gamma has been shown to have a number of opioid-like effects (Dafny, 1983). Hence, it appears that BEP may regulate IFN-gamma and IL-2-induced NK cell functions. Whether or not BEP neuronal cell transplants activate NK cell function has not previously been reported.

BEP neurons originate in the arcuate nuclei of the hypothalamus and distributed throughout the central nervous system. These neurons are involved in maintaining a variety of functions including mood, food intake, reproduction and body immune function (Boyadjieva et al., 2001; Cone et al., 2001; Smith et al., 2001; Terenius, 2000)). Lower numbers of BEP-expressing arcuate nucleus neurons have been found in various brain pathologies including schizophrenia and depression (Bernstein et al., 2002; Zangen et al., 2002). Mutation of proopiomelanocortin (POMC) gene producing BEP has been observed in obese patients (Pankov et al., 2002). The potential use of cell transplant therapy to treat these diseases and immune associated problems is difficult especially due to the lack of information regarding how BEP neurons are differentiated from neuronal progenitor cells/NSCs.

NSCs can be isolated from various parts of the brain, maintained in cultures as neurospheres in the presence of mitogens and expanded and differentiated into neurons, astrocytes and oligodendrocytes in the presence of various neurotrophic factors (Roisen et al., 2001; Roybon et al., 2004). However, the neuronal differentiation properties of NSCs depend on the region of the brain from which they have been isolated. For example, cortical neurospheres produce more dopaminergic neurons than ventral mesenchephalic neurospheres. This regional specification may make NSCs more suitable for directed differentiation of a specific neuronal phenotype.

The hypothalamus consists of several groups of hormone-secreting neurons that are critical for various neuroendocrine functions (Settle, 2000; Van den Verghe, 2000). Most of the neurons in the hypothalamus are derived from the proliferative neuroepitelium of the third ventricles (van Eerdenburgh and Swanson, 1997) and are generated during similar time in embryonic life (Markakis and Swanson, 1997). Study of cell development in the rat hypothalamus using ³H-thymidine uptake assays reveal that most of the neurons of the tuberomammillary and arcuate nuclei have late-forming starts, beginning after embryonic day 16 and continuing until birth (Altman and Bayer, 1978). However, the inductive signal involved in the generation of specific neuronal cell types from these embryonic cells has not been revealed. Using cells from the hypothalamus of 19 days old rat embryos, it has been shown that cyclic adenosine monophosphate (cAMP)-elevating agents have protective action against ethanol-induced death of □-endorphin neurons (De et al., 1994). cAMP elevating agents also have been shown to promote β-endorphin neuronal growth and neurite formation (De et al., 1994; Yang et al., 1993). These findings have raised the possibility that this neurotrophic factor can be applied to direct heterologous sets of neuronal progenitor cells forming specific neuronal phenotype. In this study, we determined whether a cAMP analog, dbcAMP, and the cAMP-elevating agent PACAP, could be used to direct the differentiation of hypothalamus-derived NSCs into functional BEP neurons. We also tested whether in vitro differentiated BEP neurons (NSC-BEP) could be used to activate NK cell function in immune suppressed fetal alcohol-exposed animals. Additionally, we tested whether the NSC-BEP cell transplants could alter the growth of prostate tumors induced by carcinogen and testosterone.

### Example 1

### Materials and Methods

Hypothalamic Neuronal Cell Cultures

Fetal brains were obtained from 17-day-old pregnant rats (Simonsen laboratories; Gilroy, Mass.). Mediobasal hypothalamic tissues from these rats were dissected out and cells from these tissues were dissociated, and mixed hypothalamic cell cultures were prepared as previously described (De et al., 1994). Neurons were separated from glial cells by filtering mixed hypothalamic cells through a 48-μM nylon mesh. Then hypothalamic cells were sedimented at 400 g for 10 min; pellets were re-suspended in HEPES-buffered Dulbecco's Modified Eagle's Medium (HDMEM, 4.5 g/l glucose; Sigma, St. Louis, Mo.); and cells were cultured into poly-ornithine coated 25-cm² tissue culture flasks (2.5 million cells/flask) in HDMEM-containing 10% fetal bovine serum (FBS) and 1% penicillin/streptomycin. On day 2, the culture medium was replaced with HDMEM containing 10% FBS, 33.6 μg/ml uridine and 13.6 μg/ml 5-fluodeoxyuridine to prevent the overgrowth of astroglial cells. All of these chemicals were obtained from Sigma. On day 3, the culture medium was replaced with HDMEM-containing serum supplement (30 nM selenium, 20 nM progesterone, 1 μM iron-free human transferrin, 5 μM insulin, and 100 μM putrescin) and 1% penicillin/streptomycin. Cells were maintained for the next 2 d with this medium. By this time, these cultures were approximately 85-90% neurons, as determined by MAP-2 positivity.

Development of Nestin-Positive Cells and Spheres

Enriched hypothalamic neurons were maintained in HDMEM, containing 10% FBS, for 3 weeks. Each week, cells were trypsinized and cultured. By the beginning of the third week, many spheres started to develop. These spheres were separated and dissociated into single cells by using trypsin/EDTA (Sigma) solution. They were then cultured in suspension or in poly-L-ornithine-coated 24-well plates (20,000 cells/well) in stem cell medium (DME F-12, lymphokine inhibitory factor (LIF), 0.1 μg/ml; L-glutamine, 10 mM; rat bFGF, 20 ng/ml; Mem amino acids solution, MAA, 0.5%; all of the chemicals were from Sigma except that bFGF was obtained from R&D Systems, Minneapolis, Minn.). Cells were cultured for 2 weeks, during which they grew and developed secondary spheres. Some of these cultures were used for immunocytochemical localization of nestin staining to determine whether they were neurospheres. These neurospheres were maintained in cultures for several months by regularly changing the medium and by splitting cells. The secondary spheres were re-suspended and cultured in poly-L-ornithine-coated 24-well plates (20,000/well; for physiological studies) or in poly-L-ornithine-coated 8-well permanox slides (1000 cells/slide; Nalg Nunc International Corp., IL; for histochemical studies). The differentiation experiments were performed by treating these cells for 1 week with PACAP (1-10 μM; SynPep) and dbcAMP (1-10 μM; Sigma) or a combination of both, and then in defined cell culture medium without the drugs for 1 week. At day 3, day 7 and day 14 the immunocytochemical, biochemical and/or real-time RT-PCR analyses were performed.

Animal Surgery/Transplantation.

Pregnant Sprague-Dawley rats were purchased and individually housed in 12-h light/12-h dark cycles (lights on at 7:00 am) and constant temperature (22° C.) throughout the study. On gestational days 11 to 21, pregnant rats were fed chow ad libitum (ad lib-fed), fed a liquid diet (BioServe Inc., Frenchtown, N.J.) containing ethanol at a level of 36% (ethanol-fed), or pair-fed an isocaloric liquid control diet (with the ethanol calories replaced by maltose-dextrin). Pups were kept with the fostered dams until postnatal day 22 and then weaned, housed by sex, and provided rodent chow meal and water ad libitum.

Differentiated cells were dissociated using 0.05% trypsin/EDTA (Invitrogen), washed and resuspended at a concentration of 20,000 cells/μl in HDME medium containing serum supplement for transplantation. Cells were placed on ice throughout the grafting session. After completion of the grafting session, cell viability was assessed using the Trypan Blue assay. Viability was greater than 90%. The composition of the differentiated cultures, with respect to the absence of undifferentiated NSCs and the presence of mature BEP-producing cells, was verified before grafting by staining for the immature neural marker nestin, and for markers of mature neurons (NeuN and MAP-2), astrocytes (GFAP), and oligodendrocytes (RIP) as well as for BEP.

Animals at forty days of age were anesthetized with sodium pentobarbital (50-70 mg/kg, i.p.; Henry Schein, Indianapolis, Ind.), and injected with 1.0 μl of stem cell suspension into each of two PVN lobes; the coordinates were set 0.5 mm from the midline, 1.8 mm behind bregma, 0.5 mm lateral of bregma, and 7.5 mm below the cortex using a 5 □l Hamilton syringe. Each injection was over a 5-minute duration. Following the injection, the cannula was left in place for 20 min as to allow the absorption of the treatment so that it would not be sucked out upon removal of the cannula. The cannula was then slowly removed in small intervals over a 10-min period. The dura was closed with 9-0 suture, muscle was re-apposed and the skin was closed with wound clips. Animals received Bupranorphin (Reckitt Benckiser; Richmond, Va.) postoperatively. Rats were injected with 30,000 IU of penicillin (Henry Schein, Indianapolis, Ind.) and placed on a heating pad for recovery. Animal surgery and care were performed in accordance with institutional guidelines and complied with NIH policy. No immune suppression was used. The animal protocol was approved by the Rutgers Animal Care and Facilities Committee.

NSC-BEP Cell Functionality In Vivo

The functionality of the NSC-BEP cells was studied in vivo by determining the changes in the expression of POMC and CRH mRNA in the PVN following systemic administration of LPS between the animals with NSCs transplants and sham-transplants. We also determined the LPS-induced changes in the expression of POMC and CRH mRNA in the PVN in control animals that were untreated with alcohol during fetal life. We used the 100 μg/kg dose of LPS for a period of 3 h (which was found to be an effective dose; Chen et al., 2006) to determine the changes in the hypothalamic CRH and BEP responses of the NSCs transplants or control transplants (cortical cell transplants). From the brains of these animals, we collected PVN by punching. The tissues were used to determine POMC mRNA and CRH mRNA levels by real-time RT-PCR methods or BEP and CRH levels by radioimmunoassay.

Immunohistochemistry

Cell cultures were fixed in 4% paraformaldehyde for 30 min and then in 70% ethanol for an additional 30 min. The immunocytochemistry was performed by using a Quick Kit (Vector Laboratories Inc., Burlingame, Calif.) and following the instructions provided in the kit. Cells were incubated with primary antibodies overnight at 4° C. Primary antibodies used were monoclonal antibodies for nestin (BD Pharmingen; San Jose, Calif. 1 μg/ml), vimentin (clone V9, mouse ascites fluid, 0.22 μg/ml; Sigma; 1:40), α-internexin (Santa Cruz Biotechnology, Santa Cruz, Calif.; 1 μg/ml), MAP2 (2A+2B, clone AP-20, mouse ascites fluid, 0.72 μg/ml; Sigma), β-tubulin (type III, clone SDL.3D10, mouse ascites fluid, 0.30 μg/ml, Sigma), GFAP (clone G-A-5, 45 μg/ml, Sigma), NF-M (145 kDa, 5 μg/ml, Chemicon International, Temecula, Calif.), TH (IgG1, 1:500; BD Biosciences), polyclonal primary rabbit antibody for β-endorphin (1:1000; Peninsula Laboratories, San Carlos, Calif.), GnRH (1:500; Chemicon) and NPY (1:500; Peninsula Laboratories). The secondary antibody used to react with mouse primary antibodies (Nestin, MAP2, type III β-tubulin, NF-L, NF-M and alpha-internexin) was Alexa Fluor 488 donkey anti-mouse IgG, (4 μg/ml; Molecular Probes, Eugene, Oreg.) and with rabbit primary antibody (β-endorphin) was the Alexa Fluor 594 donkey anti-rabbit IgG (H+L) (4 μg/ml; Molecular Probes). Both of these secondary antibodies failed to stain the NSC or differentiated cells in the absence of a primary antibody. Some of the cell-containing chambers were dried and mounted using DAPI-containing Mounting Medium (H-1200; Vector Laboratories Inc.). Fluorescent images were captured with a Cool SNAP-pro CCD camera coupled to a Nikon-TE 2000 inverted microscope. Images were processed with Adobe Photoshop 7.0.

Radioimmunoassay

The immunoreactive β-endorphin levels in culture medium samples were measured by a radioimmunoassay system (De et al., 1994). All the samples were dried and reconstituted in assay buffer and measured in duplicate in a single assay. The minimum amount of β-endorphin detectable was 3 pg/tube.

Real-Time RT-PCR Analysis

Expression levels of POMC mRNA in NSCs and differentiated cells were measured by a quantitative real-time RT-PCR (TaqMan assay) on an ABI PRISM 7700 Sequence Detector (PerkinElmer Applied Biosystems, Foster City, Calif.) as described by us previously (Chen et al., 2004). This assay is based on the 5′ nuclease activity of Taq DNA polymerase for fragmentation of a dual-labeled fluorogenic hybridization probe and was performed following the manufacturer's instructions. Total RNA was isolated from the differentiated cultures using an RNeasy Mini Kit (Qiagen, Valencia, Calif.) and following the manufacturer's instructions. The genomic DNA was removed by DNase I treatment. Total RNA (1 μg) was subjected to first-strand cDNA synthesis using the Superscript First-Strand Synthesis System for RT-PCR (Invitrogen, Carlsbad, Calif.). cDNA was subjected to real-time RT-PCR. The expression of POMC mRNA was detected using a POMC gene-specific primer pair and probe (TaqMan Gene Expression Assay, Rn00595020_m1; Applied Biosystems; Foster City, Calif.). PCR amplifications were performed by incubating at 50° C. for 2 min and then 95° C. for 10 min followed by 40 cycles at 95° C. for 15 sec and 60° C. for 1 min. The relative quantity of mRNA was calculated by relating the PCR threshold cycle obtained from the tested samples to relative standard curves degenerated from a serial dilution of cDNA prepared from the total RNA. The POMC mRNA level in each sample was normalized with the level of GAPDH mRNA which was measured by a control reagent (PerkinElmer Applied Biosystems).

Immune Cell Response to NSC-BEP Challenge

NK cell respond to an immune challenge after 3 weeks following PVN transplants of NSC-BEP cells or control cortical cells in 40 days old alcohol-fed, ad lib-fed and pair-fed female rats. The NK cell response was determined by measuring the NK cell cytolytic activity in the PBMC and spleen, and cytokines (IFN-gamma, TNF-alpha) levels in the plasma.

Tissue and Plasma Sample Collection for NK Assay

At the end of the experiment, animals were decapitated and spleens were obtained aseptically. Splenocytes were isolated from whole spleens of these rats and processed for isolation of NK cells by magnetic separation using negative selection procedures as we have described previously (Dokur et al., 2005; Arjona et al., 2006). Cells in the negative fraction are enriched NK cells. Cells in positive selection are non-NK-splenocytes. The viability of enriched NK cells range above 90-95%. For PBMC isolation, blood was centrifuged at 1500 g for 20 minutes to remove the plasma. The cell pellet was resuspended in Hanks' balanced salt solution (Gibco BRL/Invitrogen, Carlsbad, Calif., USA) in a volume of the same as the original volume of the centrifuged sample and the cell suspension was carefully layered over the top of 5 ml of 95% Ficoll (Amersham Pharmacia) in a 15 ml Falcon tube. The tubes were centrifuged for 40 minutes at 1500 g and the white cell layer was collected using a Pasteur pipette. PBMCs were rinsed with cold Hanks' balanced salt solution and used for determination of NK cell cytolytic activity.

NK Cell Cytolytic Assay

Cytolytic activity was determined from quadruplicate measures of effector to target ratios of 200:1, 100:1, 50:1 and 25:1 against chromium-labeled YAC-1 lymphoma cells in a 4-h assay as previously described (Boyadjieva et al., 2001). The Lytic unit was calculated from the cytolytic data at 10% cytolytic activity for 10⁶ effector cells according to Pross et al., (1981). Data will also be expressed as lytic units/NK cell determined by flow cytometry. Expression of the data in this way will allow us to interpret whether the absolute lytic activity of the NK cells is modulated or whether changes in activity are due to alteration in the number of NK cells.

NK Cell Cytolytic Factors and Cytokine Production

Cellular content of granzyme B, perform, IFN-gamma and TNF-alpha levels were assayed by western blot as described by us previously (Arjona et al., 2004; Dokur et al., 2003). Plasma and media contents of INF-gamma were measured by ELISA (Amersham Biosciences; Piscataway, N.J.). Relative protein levels were calculated as a percentage of the maximum value observed in each blot. These assays are routinely used in our laboratory (Arjona et al., 2004; Dokur et al., 2003).

NSC-BEP Cell Transplants and the Growth of Prostate Tumors Induced by Carcinogen and Hormone in Rats

We conducted a study to determine the effects of the cell transplants on carcinogen and hormone induced prostate tumor growth. Carcinogen and hormone exposure was carried out using the well-established MNU and testosterone treatment protocols published previously (Arunkumar et al., 2006). In this experiment, control fed male rats were transplanted with NSC-BEP cells, cortical cells or NSC cells in both PVN and then were injected interperitoneally (i.p). with cyproterone acetate (50 mg/kg body wt.) (Sigma Chemicals) for 21 consecutive days. One day after the last dose of cyproterone acetate, rats received daily i.p. injection of 100 mg testosterone propionate/propylene glycol for 3 d. One day after the last testosterone propionate injection all rats received a single i.v. dose (50 mg/kg body wt.) of MNU (dissolved in saline at 10 mg/ml), through the tail vein. One week after MNU administration, rats received daily i.p. injection of 2 mg/kg body wt. testosterone propionate/kg body wt. for 60 d. After the treatment period rats were killed, prostate was removed from the adhering connective tissue, washed several times with physiological saline, weighed accurately, fixed with 10% neutral buffered formalin and stained with hematoxyline and eosin for determination of tissue histopathology.

Statistics

The means±standard errors of the data were determined and are presented in the text and figures. Data were analyzed using one-way ANOVA. The differences between groups were determined using the Student-Newmann-Keuls test. A value of p<0.05 was considered significant.

### Results

caMP-Elevating Agents Increased Differentiation of Hypothalamic NSCs to β-Endorphin Neurons in Culture

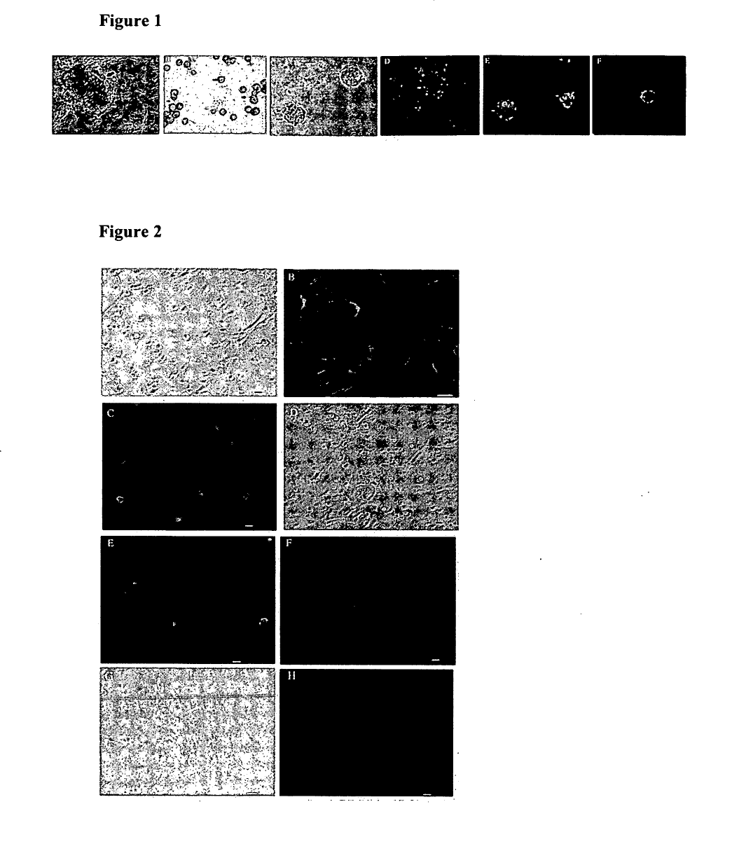

To begin to examine the capacity of NSCs to generate β-endorphin neurons, we purified neurons from embryonic hypothalamic tissues and grew neurospheres in cultures using stem cells maintaining medium. These neurospheres were maintained in cultures for a period of 2 weeks in the presence or absence of added factors including basic fibroblast growth factor (bFGF). Upon dissociation, they developed secondary neurospheres and formed aggregates that expressed nestin and vimentin (FIG. 1), protein markers of the immature uncommitted phenotype (Lendahl et al., 1990; Shaw, 1998). The neurosphere was considered NSC. These neurospheres can be maintained in culture for several months by regularly changing medium and splitting cells.

Whether PACAP and dbcAMP affect differentiation of these NSCs into neurons in serum-free neuronal cell-maintaining medium was determined. Within a period of 3 d, many neurospheres start forming single cell with various shapes (FIG. 2A), many of these cells showed significant amount of vimentin (FIG. 2B), and α-internexin immunoreactivity (FIG. 2C) which is a marker of early neuronal phenotypes (Kaplan et al., 1990; Carden et al., 1987). After 1 week of PACAP and dbcAMP treatments, NSCs began to show filamentous structures (FIG. 2D) and to express neurofilament (NF)-M (FIG. 2E), a neuronal marker (Carden et al., 1987), indicating that the neuronal progenitor cells had begun to differentiate into neuronal phenotypes by this time. Characterization of the neuronal phenotypes by the immunohistochemical method revealed that many of these cells were expressing β-endorphin (FIG. 2F). Neuronal stem cells that were maintained in neuronal-cell maintaining media without PACAP and dbcAMP did not show many cells with filamentous structures (FIG. 2G) nor did they show any β-endorphin-staining (FIG. 2H), suggesting the possibility that the cAMP activating agents are necessary for NSCs differentiation to β-endorphin neurons.

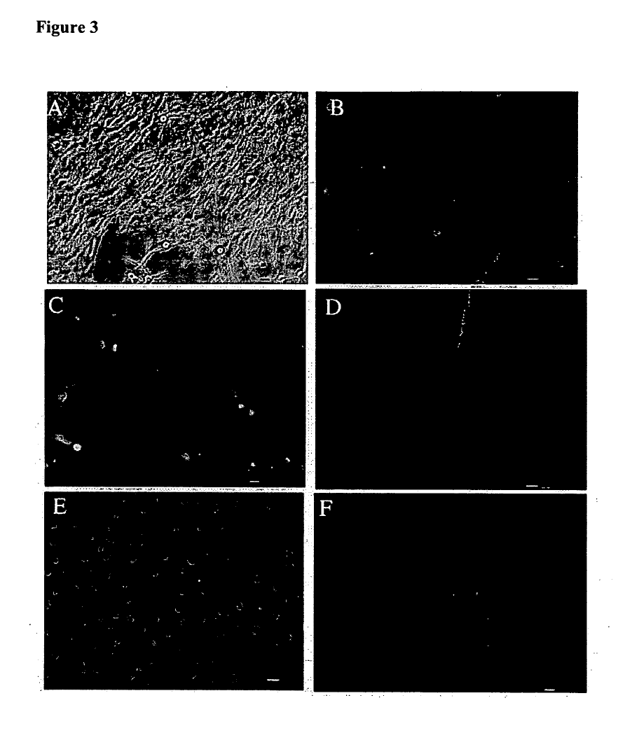

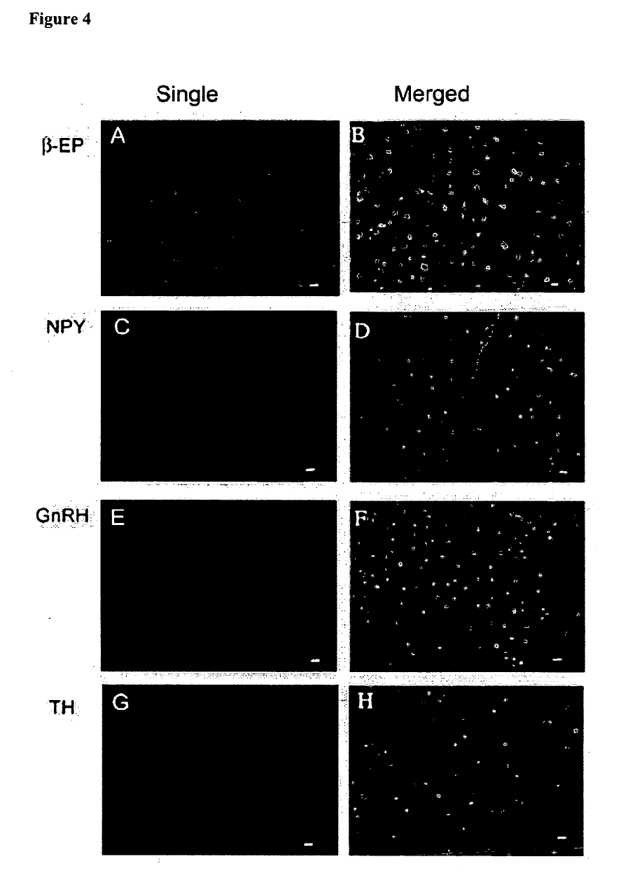

These NSCs were further maintained in serum-free defined neuronal cell culture medium without cell differentiating factors for a period of 1 week in order to determine the permanency of the PACAP/dbcAMP effects on NSC differentiation. By the end of this treatment, all of these cells had neuron-like appearance (FIG. 3A) and expressed neuronal markers (Evans et al., 2002) MAP2 (FIG. 3B) and type III β-tubulin (FIG. 3C) but not astrocyte cell marker (Raju et al., 1981) glial fibrillary acid protein (GFAP; FIG. 3D), suggesting that all NSCs were now differentiated into neurons. These cells also stained for BEP (FIG. 3E). Control experiment with excess antigen verified BEP immunostaining on differentiated NSCs (FIG. 3F). The BEP antibody we used also stained cells producing this peptide in the hypothalamus (FIG. 4A). Further characterization of the differentiated NSCs revealed that 100 percent of these cells are BEP immunopositive (FIG. 4B; note that all the cells identified by the blue color nuclear staining with DAPI also stained for red color BEP). However, these differentiated NSCs did not stain for neuropeptide Y (NPY; FIGS. 4C and D), gonadotropin hormone-releasing hormone (GnRH; FIGS. 4E and F), or tyrosine hydroxylase (TH; an enzyme produces catecholamine including dopamine; FIGS. 4G and H). These are some of the major peptides in the hypothalamus that are positively regulated by the cAMP and PACAP system (Li et al., 1996; Olcese et al., 1997; Hansel et al., 2001; Mizuno et al., 1998; Reglodi et al., 2004). Hence, activation of the cAMP system may lead to differentiation of NSCs to primarily BEP neurons.

caMP Agent-Induced Differentiated Neurons Had β-Endorphin Neuronal Function

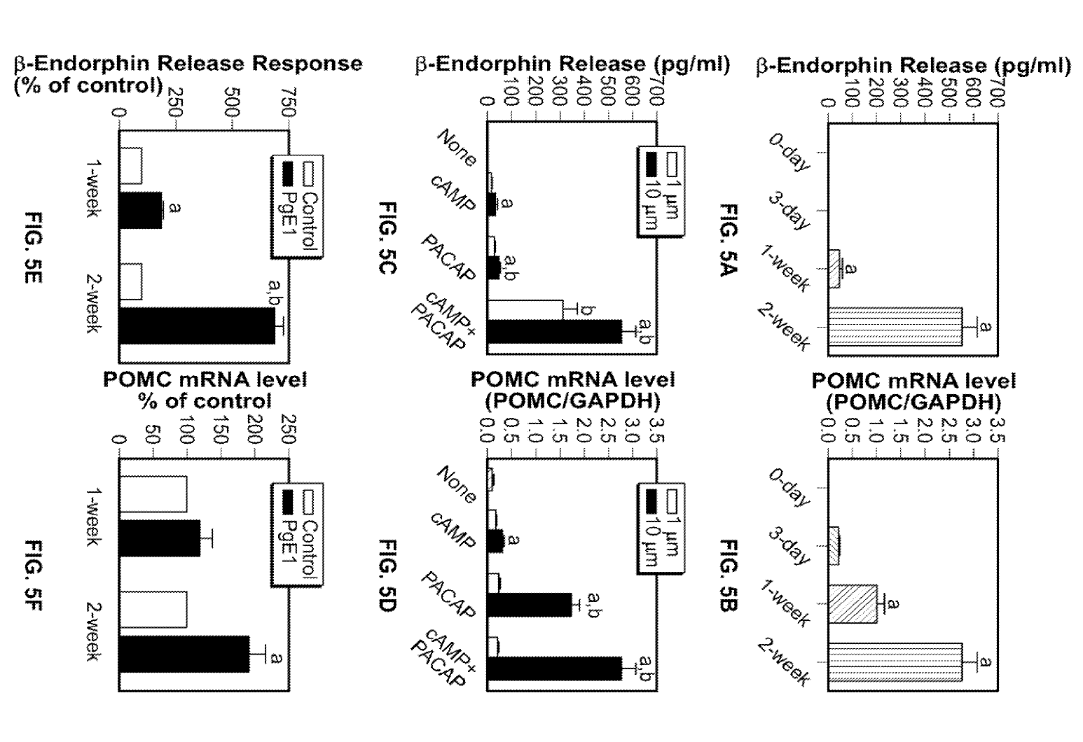

To clarify whether the immunoreactive characteristic of differentiated neuronal progenitor cells reflect their functions, we first studied the dynamic of basal secretion of β-endorphin during the period of differentiation in culture. In agreement with the immunohistochemical data, FIG. 5A shows that NSCs at the end of PACAP and dbcAMP treatment at 1 week secreted moderate amounts of BEP in the media, but secreted 10-12-fold larger amounts of the peptide in the media even in the absence of the cAMP elevating agents at 2 weeks. Additionally, the amount of BEP released from these cells showed dose-dependency and additive effects of PACAP and dbcAMP (FIG. 5C). The control cells, which were not treated with PACAP or dbcAMP (0 day), showed no detectable amount of BEP release.

The peptide BEP is processed from a precursor protein proopiomelanocortin (POMC) from arcuate neurons in vivo (Castro and Morrison, 1997). Whether the in vitro differentiated BEP neurons express POMC mRNA in a fashion similar to that of BEP release was determined. The expression patterns of POMC mRNA levels showed time-dependency and dose-dependency on PACAP and/or dbcAMP and resembled those patterns of BEP release during differentiation (FIGS. 5B and D). Together these data indicate that cAMP agents promoted differentiation of NSCs into BEP-producing cells.

After determining that in vitro differentiated neurons express POMC and release BEP in medium, we studied their ability to respond to the neuromodulator-like prostaglandin E1 (PGE1), which is known to elevate BEP release from hypothalamic cells (Boyadjieva and Sarkar, 1997). FIGS. 5E and F show that, at 1 week, PGE1 only moderately enhanced BEP release and produced no effect on POMC expression from the group of cells that differentiate in the presence of the combination of PACAP and dbcAMP. However, at 2 weeks the BEP-release response and POMC-expression response to PGE1 were significantly increased. These results indicate that the differentiated neurons have the functional capacity to produce and release BEP and to respond to activators such as PGE1.

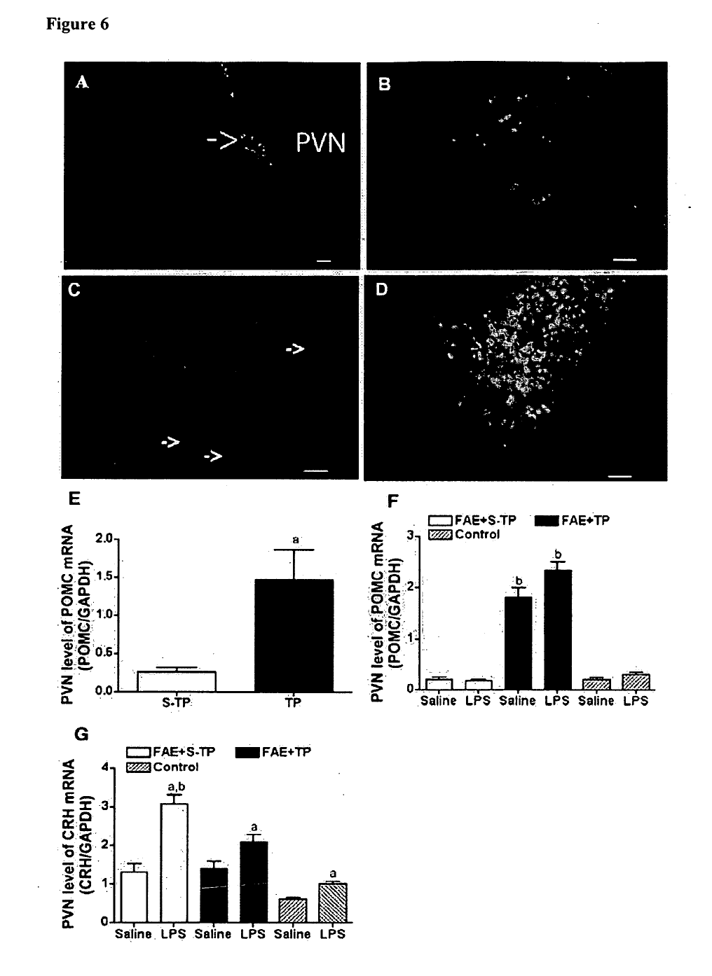

To determine whether the differentiated NSCs maintain their neuronal phenotype in vivo, we labeled these cells with bromodeoxyuridine (BrdU) and transplanted these cells into one of the lobe of PVN of the hypothalamus that contains very few BEP cell bodies. Two weeks after transplantation, these cells remained at the site of transplantation in the PVN (FIGS. 6A and B) and showed immunostaining for BEP (FIGS. 6C and D). By determining levels of POMC mRNA in the PVN, we found that expression of this gene was higher by a factor of 6 in the lobe of PVN where the differentiated cells were transplanted than in the contralateral lobe of the PVN that contained sham-transplants (FIG. 6E).

The availability of functional BEP neurons from NSCs led us to conduct a replacement therapy study to determine the effect of NSC-BEP neurons on CRH neuronal response in male rats exposed to ethanol during the prenatal period (see Arjona et al., 2006, for methods of animal preparation). Rats exposed to ethanol during the prenatal period are known to demonstrate CRH hyperresponsiveness to an immune challenge (Lee et al., 2000; Taylor et al., 1988). CRH hyperresponsiveness in alcohol-fed rats has shown to be due to low BEP neuronal function (Sarkar et al., 2007). Hence, we investigated the CRH neuronal response to these NSC-BEP transplants by infusing these cells into one PVN lobe and infusing control medium into the other PVN lobe of the alcohol-fed rats. We then compared the changes in the expression of POMC and CRH mRNA levels between the two sides of the PVN of each animal following systemic administration of LPS. As shown in FIG. 6F, LPS moderately increased the POMC levels in the PVN containing the NSC-BEP neurons but did not affect PVN levels of POMC in the contralateral side without cell transplants in alcohol-fed animals. LPS was also ineffective in altering POMC levels in the PVN of control-fed rats. LPS increased CRH mRNA levels in the PVN of both non-transplanted alcohol-fed and control-fed rats, but the magnitude of the CRH response to LPS was higher in alcohol-fed rats (FIG. 6G). LPS increased the level of CRH mRNA in the PVN infused with NSC-BEP neurons or with control media in alcohol-fed rats. However, the response of PVN lobe side containing NSC-BEP neurons was much lower than the response of the PVN side treated with control medium. These results indicate that NSC-BEP cells were able to reduce the CRH hyperresponsiveness to LPS in alcohol-fed rats.

NSC-BEP Cell Transplants Activate NK Cell Function

Previously we have shown that administration of BEP peptide into the PVN increases the cytolytic function of splenic NK cells (Boyadjieva et al., 2001) by suppressing the inhibitory action of CRH on splenic NK cells via sympathetic neurons (Boyadjieva et al., 2006). Since NSC-BEP cell transplants can prevent CRH function, we investigated whether NSC-BEP cell transplants activate NK cell function. We studied this by transplanting NSC-BEP cells or control cells in immune-deficient neonatally alcohol-fed male rats (Arjona et al., 2006; Zhang et al., 2005) during the adult period in the one PVN or both PVNs in alcohol-fed and control-fed rats. As a control for NSC-BEP cells, we used cortical cells (Noh and Gwag 1997). We used neonatally alcohol-fed rats because these rats are immune-deficient and show reduced NK cell function (Arjona et al., 2006).

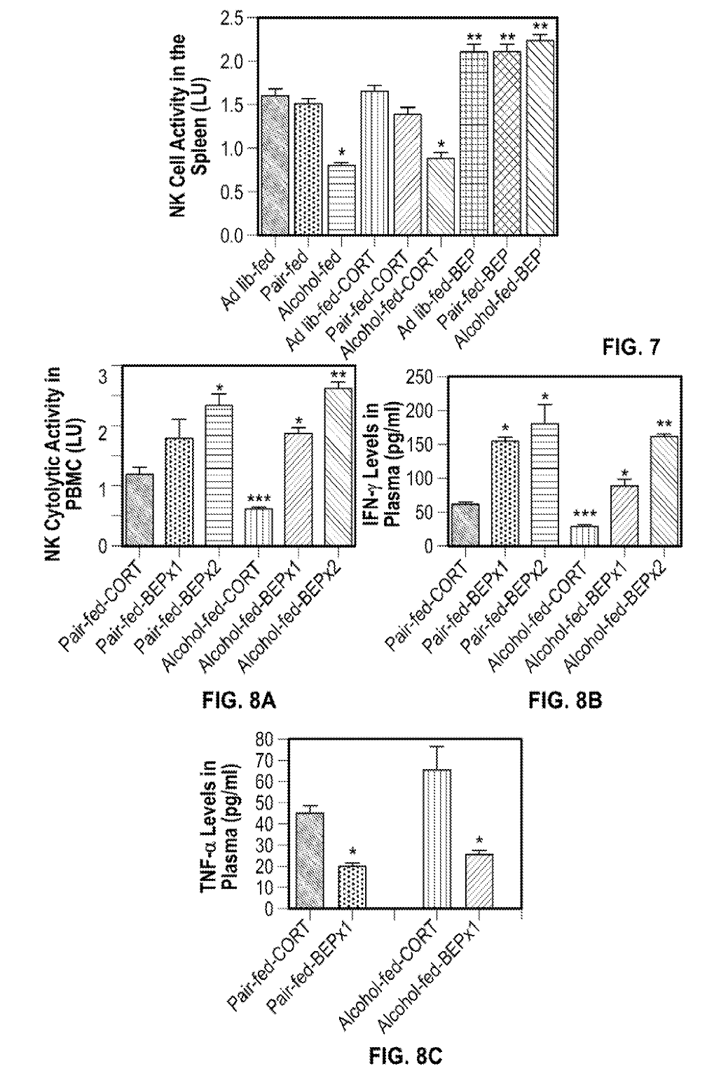

Determination of NK cell functions revealed that NSC-BEP transplants significantly increased NK cell cytolytic activity in the spleens (FIG. 7) in control-fed and alcohol-fed rats. The NK cell activation effect of NSC-BEP cells was higher when transplanted in both PVNs as compared to only one PVN. Bilateral NSC-BEP cell transplants in both PVNs were also able to increase cytolytic function of NK cells derived from peripheral blood mononuclear cells (PBMC; FIG. 8a) and levels of IFN-gamma in peripheral plasma (FIG. 8b) of alcohol-fed and control-fed animals. However, NSC-BEP cell transplants decreased TNF-alpha levels in the plasma of alcohol-fed and control-fed animals (FIG. 8C). The levels of both basal and LPS-induced NK cytolytic function and IFN-gamma mRNA levels in splenic tissues of alcohol-fed animals transplanted with NSC-BEP neurons in the PVN were higher than those levels in alcohol-fed animals with sham transplants in the PVN. These results indicate that NSC-BEP cell transplants are effective in activating NK cell functions.

NSC-BEP Cell Transplants Reduce the Growth of Prostate Tumors Induced by Carcinogen and Hormone in Rats

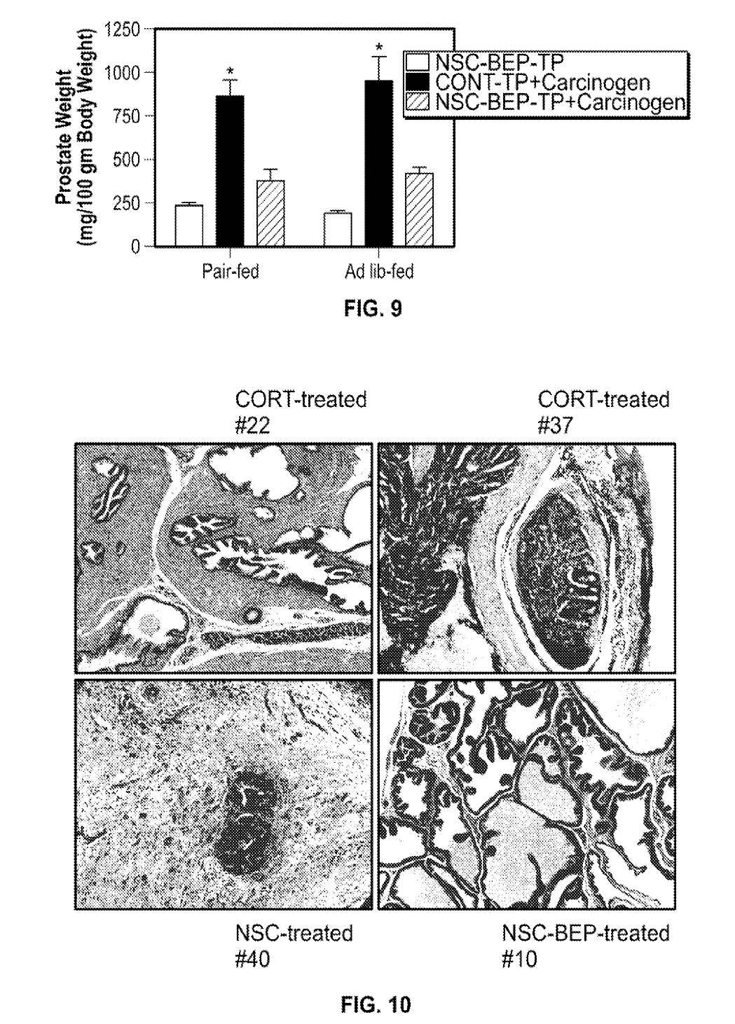

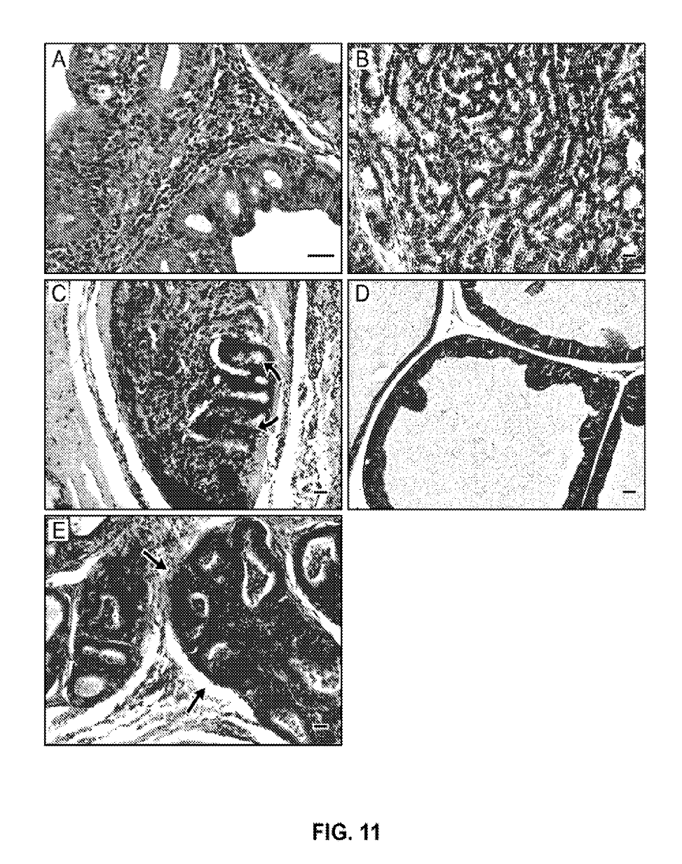

We conducted an experiment to determine the effects of the NSC-BEP cell transplants on carcinogen and hormone induced prostate tumor growth using well-established MNU and testosterone treatment protocols (Arunkumar et al., 2006). In this experiment, control fed male rats were transplanted with NSC-BEP cells, cortical cells or NSC cells in both PVN and then were treated with carcinogen and hormones. After the treatment period, rats were killed, prostate was removed from the adhering connective tissue, washed and weighed. The wet weights of prostate/kg of body weight were 3-4-fold lower in animals treated with NSC-BEP cells than in cortical cells or NSC cells (FIG. 9). A limited characterization of histopathology revealed substantial neoplasia in all the prostrates of four control cell transplanted animals but no neoplasia in the prostate of four NSC-BEP cells transplanted animals (FIG. 10). This data indicates that NSC-BEP cell therapy has the potential to prevent tumor cell growth.

Discussion

Our main finding was that neuronal progenitor cells can be generated from the rat embryonic hypothalamic tissues and propagated by cAMP-elevating agents to produce BEP neurons in cultures. Like in vivo, these cells go on to produce and secrete BEP and respond positively to the neuromodulator challenge. Early in mammalian brain development, NSCs express vimentin and nestin intermediate filament proteins (Lendahl et al., 1990; Shaw, 1998). As development progresses, these cells divide and differentiate to produce neuronal or glial lineage. Here we showed that cultures derived from embryonic rat hypothalamic cells generated nestin-positive neuronal progenitor cells. These cells grew and generated secondary nestin-positive spheres when primary spheres were mechanically dissociated and cultured under the same experimental conditions.

bFGF has previously been shown to induce or enhance the proliferation of neurospheres and neuronal stem cells (Vescovi et al., 1993). In our study, we used bFGF to generate and develop a colony of undifferentiated cells, which expressed nestin, or vimentin. Here we showed that the NSCs isolated in vitro from rat embryonic hypothalamic neurons responded to bFGF under serum-free conditions to give rise to clonal aggregates of undifferentiated neurospheres. The differentiation of hypothalamic-derived neuronal precursor cells to neurons was initiated by 3 days after PACAP and/or dbcAMP treatments since they expressed the marker for immature neurons-α-internexin (Kaplan et al., 1990; Carden et al., 1987). These cells differentiated into neuronal phenotypes by the end of a week of PACAP and/or dbcAMP treatments since they expressed NF-M. This neurofilament protein is a well-known neuronal marker for neurodifferentiation (Carden et al., 1987). In addition, 1 week of post PACAP and cAMP treatments, these treated cells expressed the neuronal markers MAP2 and type III β-tubulin but not the astrocyte marker GFAP (Evans et al., 2002; Raju et al., 1981). This is consistent with the conclusion that combination treatments of PACAP and dbcAMP activated the development and maturation of precursor hypothalamic spheres to the pure population of neurons.

PACAP belongs to a peptide family that includes secretin, glucagons, growth hormone-releasing factor, and vasoactive-intestinal peptide (Sherwood et al., 2000). Recent data suggest that PACAP exerts developmental actions. PACAP gene expression and PACAP immunoreactivity are widely distributed in neurons within the embryonic and neonatal rat brain (Nielsen et al., 1998). Activation of PACAP receptors regulates the proliferation of the developing neuroblasts in vitro and in vivo (Lee et al., 2001; DiCicco-Bloom et al., 2000). PACAP and its receptors are expressed in embryonic neural tubes, where they appear to regulate neurogenesis (Lee et al., 2001). Our results demonstrated that PACAP promoted the differentiation of embryonic stem cell neurons. The results reported here also provide the first evidence that dbcAMP interacts with PACAP in the induction of differentiated mature neurons. Moreover, the results here demonstrated for the first time that the combination of PACAP and dbcAMP promote differentiation and functional maturation of BEP neurons.

We have previously shown that dbcAMP acts as a neurotropic factor for immature BEP neurons (De et al., 1994). Also, studies of transduction pathways identified that the adenyl cyclase-cAMP system is an important second messenger system in the regulation of hormone secretion and POMC gene regulation (Lundblad and Roberts, 1998). Since PACAP is a potent inducer of cellular levels of cAMP in developing neurons (DiCicco-Bloom et al., 2000), the interactive actions between this peptide and dbcAMP we observed support that the cAMP signaling system regulates early differentiation of BEP neurons.

The availability of functional BEP neurons from NSCs also might be useful for replacement therapy study. We found that the NSC-derived BEP neurons have the ability to produce POMC for a minimum period of two weeks after intra-PVN transplantation. Exogenous BEP has been shown to inhibit CRH neuronal function (Boyadjieva et al., 2006; Plotsky, 1986). BEP and CRH are known to regulate immune functions (Boyadjieva et al., 2001 and 2006; Irwin et al., 1988), and these peptides and their mRNA levels are increased following LPS treatments or other immune challenges (Chen et al., 2006; Lee et al., 2000; Taylor et al., 1988)). In this study, we show that the level of POMC mRNA in BEP neuronal transplants was moderately, but not significantly, increased following the LPS challenge. However, even in this moderate elevation of POMC, the CRH mRNA response to LPS was markedly decreased in animals with the BEP neuronal transplant in the PVN. Hence, the NSC-derived BEP neurons are reducing the CRH neuronal ability to respond to the LPS challenge. These results indicate that NSC-derived BEP neurons maintain functionality when they are transplanted in vivo.

Since NSC-BEP cell transplants can prevent CRH function, we investigated whether NSC-BEP cell transplants activate NK cell function. The NK cell activation effect of NSC-BEP cells was higher when transplanted in both PVNs as compared to only one PVN. Bilateral NSC-BEP cell transplants in both PVNs were also able to increase NK cytolytic function and plasma levels of IFN-gamma in alcohol-fed animals. However, NSC-BEP cell transplants decreased TNF-alpha levels in the plasma of alcohol-fed and control-fed animals. The levels of both basal and LPS-induced IFN-gamma mRNA levels in splenic tissues of alcohol-fed animals transplanted with NSC-BEP neurons in the PVN were higher than those levels in alcohol-fed animals with sham transplants in the PVN. However, both the basal and LPS-induced TNF-alpha mRNA were similar in spleen tissues of alcohol-fed animals transplanted with or without NSC-BEP neurons. These results suggest that NSC-BEP cell transplants are effective in activating NK cell functions.

The ability of NSC-BEP to increase the NK cell activity was correlated with the ability of this cell transplant to inhibit prostate tumor growth and progression. These data indicate that NSC-BEP cell therapy has the potential to increase the body's innate immunity to prevent growth and progression of cancer cells.

Pituitary adenylate cyclase-activating peptide (PACAP), a cAMP-activating agent, is highly expressed in the hypothalamus during the period when many neuroendocrine cells become differentiated from the neural stem cells (NSCs). Activation of the cAMP system in rat hypothalamic NSCs differentiated these cells into beta-endorphin (BEP)-producing neurons in culture. When these in vitro differentiated neurons were transplanted into the paraventricular nucleus (PVN) of the hypothalamus of an adult rat, they integrated well with the surrounding cells and produced BEP and it's precursor gene product, proopiomelanocortin (POMC). Animals with BEP cell transplants demonstrated remarkable protection against carcinogen induction of prostate cancer. Unlike carcinogen-treated animals with control cell transplants, rats with BEP cell transplants showed rare development of glandular hyperplasia, prostatic intraepithelial neoplasia (PIN) or well-differentiated adenocarcinoma with invasion following N-methyl-N-nitrosourea (MNU) and testosterone treatments. Rats with the BEP neuron transplants showed increased natural killer (NK) cell cytolytic function in the spleens and peripheral blood mononuclear cells (PBMC), elevated levels of anti-inflammatory cytokine interferon-gamma IFN-gamma and decreased levels of inflammatory cytokine tumor necrosis factor-alpha (TNF-alpha) in plasma. These results identify a critical role for cAMP in the differentiation of BEP neurons and revealed a novel role of these neurons in combating the growth and progression of neoplastic conditions like prostate cancer, possibly by increasing the innate immune function and reducing the inflammatory milieu.

### Introduction

The hypothalamus consists of several groups of hormone-secreting neurons that are critical for various neuroendocrine functions (1). Most of the neurons in the hypothalamus are derived from the proliferative neuroepithelium of the third ventricle (2). A study of cell development in the rat hypothalamus, using ³H-thymidine uptake assays, revealed that most of the neurons of the tuberomammillary and arcuate nuclei have late-forming starts, beginning after embryonic day 16 and continuing until birth (3). However, the inductive signal involved in the generation of specific neuronal cell types from these embryonic cells has not been identified. Using cells from the hypothalamus of rat embryos, it has been shown that cAMP-elevating agents protect against ethanol-induced death of BEP neurons (4). These findings have raised the question of whether this neurotrophic factor can be used to direct heterologous sets of NSCs into specific neuronal phenotype.

BEP neuronal cell bodies are primarily localized in the arcuate nuclei of the hypothalamus, and its terminals are distributed throughout the central nervous system. These neurons are involved in maintaining a variety of functions including stress regulation and immune functions. Abnormalities in BEP neuronal function are correlated with various pathologies. For example, lower numbers of BEP neurons have been found in the postmortem brains of patients with schizophrenia and depression and a reduced BEP production due to POMC gene mutation has been observed in many obese patients. It is noteworthy that a higher incidence of cancers and infection has been found under these pathological conditions. Furthermore, the endogenous function of BEP neurons is reported to be reduced in cancer patients. Therefore, we hypothesized that increased BEP neuronal activity might be beneficial to control the growth of tumors. In this study, we examined whether a cAMP analog, dbcAMP, and PACAP can be used to direct the differentiation of hypothalamus-derived NSCs into functional BEP neurons in vitro, and we determined the functionality of differentiated cells in vivo by transplanting them into the hypothalamus of male rats and evaluating their effects on carcinogen-induced prostate tumors and immune functions.

### Example 2

### Results

cAMP-elevating agents increased differentiation of hypothalamic NSCs to BEP neurons in cultures. To examine the capacity of NSCs to generate BEP neurons, we purified neurons from embryonic hypothalamic tissues and grew neurospheres in cultures using stem cell-maintaining medium. It was determined whether or not PACAP and dbcAMP differentiate NSCs into neurons. An initial screening of the response of various doses (0.1-10 μM) of PACAP and dbcAMP alone revealed a moderate effect of these agents on neurosphere differentiation, since many cells remained as neurosphere like structures. However, using a combined treatment of 10 μM concentrations of PACAP and dbcAMP, we found many neurospheres started forming single cells with various shapes within a 3-day period. Many of these cells expressed significant amounts of vimentin and α-internexin immunoreactivity, which are markers of early neuronal phenotypes. After 1 wk of PACAP and cAMP treatment, NSCs began to show filamentous structures and to express neurofilament (NF)-M, a neuronal marker, indicating that the NSCs had begun to differentiate into neurons. Determination of the phenotype revealed that many of these cells (50-60%) were expressing BEP at this stage of differentiation. NSCs that were not treated with PACAP and cAMP showed no staining for BEP (IH).

The differentiated NSCs were further maintained in a defined-neuronal cell culture medium for a period of 1 wk in order to determine the permanency of the PACAP/cAMP effects. By the end of this treatment, all of these cells had a neuron-like appearance, and they expressed neuronal markers MAP2 and type III β-tubulin, but not the astrocyte cell marker GFAP, suggesting that all NSCs were now differentiated into neurons. These cells also stained for BEP. A control experiment with excess antigen verified the specificity of BEP immunostaining in differentiated NSCs (S1A). The staining of BEP for these cells merged well with the nuclear staining for DAPI (S1B), suggesting that the majority of NSCs is differentiated into BEP cells. However, these differentiated NSCs did not stain for neuropeptide Y, gonadotropin hormone-releasing hormone or tyrosine hydroxylase (S1C-E). These are some of the major peptides in the hypothalamus positively regulated by the cAMP and PACAP system. Hence, activation of the cAMP system rusult in differentiation of NSCs to primarily BEP neurons.

cAMP agent-induced differentiated neurons produced and released BEP. To clarify whether the differentiated NSCs replicate the functions of BEP neurons, we studied the dynamics of basal secretion of BEP during the period that the NSCs were differentiating in culture as well as after the completion of differentiation. In agreement with the immunohistochemical data, results showed immediately after the 1 wk treatment with PACAP and cAMP, NSCs secreted moderate amounts of BEP into the media. Furthermore, 1 wk after the weeklong treatment with PACAP/cAMP, NSCs secreted an amount of peptide in the media 10-12-fold greater than that which was produced immediately after the treatment. Like BEP release, the POMC mRNA levels in the cells were markedly increased at this time. Additionally, the amount of hormone released from these cells by PACAP and cAMP as a result of the amount of BEP neuron differentiation showed dose-dependency and synergistic effects when the two differentiating agents were combined. The expression patterns of POMC mRNA resembled the patterns of BEP released during PACAP and cAMP-induced differentiation. We also studied the ability of differentiated BEP neurons to respond to prostaglandin E1 (PGE1), which is known to elevate BEP release from hypothalamic cells. Results show that BEP release and POMC-expression of the differentiated neurons were increased by PGE1. These results suggest that the differentiated neurons produce and release BEP in culture.

To determine whether the differentiated neurons maintained their neuronal phenotype in vivo, they were labeled with bromodeoxyuridine (BrdU) and transplanted into one lobe of the PVN of the hypothalamus, a site containing very few BEP cell bodies. Two weeks after the transplantation, these cells remained at the site of transplantation in the PVN and showed immunostaining for BEP. By determining levels of POMC mRNA in the PVN, we found that the expression of this gene was higher by a factor of 6 in the PVN where the BEP cells were transplanted than the lobe of the PVN into which nonviable BEP cells had been implanted. Determination of BEP protein levels in the hypothalamus and in plasma revealed that the transplants increased levels of this protein in the site of transplants but not in the circulation.

cAMP agent-induced differentiated BEP neurons reduced MNU-induced prostate tumors. To determine the effect of BEP cell transplants on tumor growth and development, we implanted BEP cells in both lobes of the PVN of male rats and treated them with MNU and testosterone as previously described. For the control transplant, we used viable, non-BEP-producing fetal rat cortical cells rather than the non-viable BEP cells for a long-term transplant study. The viability of BEP cell transplant was tested by determining the plasma corticosterone response to LPS in the animal prior to sacrifice. We hypothesized that if BEP cell transplants were functional they would have the ability to inhibit LPS-induced CRH release and therefore corticosterone release in the circulation. BEP neurons have been shown to inhibit CRH, which regulates the plasma level corticosterone. We found that LPS increased plasma corticosterone level in rats treated with control transplants and MNU and testosterone (saline-285±15; LPS-478±20; N=11-12; P<0.05) but not in rats treated with BEP cell transplants without MNU and testosterone (saline-305±15; LPS-328±14; N=5-6) or with MNU and testosterone (saline-355±35; LPS-398±20; N=8-9), suggesting that BEP neuron transplants were functional until the end of the treatment.

We found that total weight of prostates in rats treated with BEP neuron transplants and carcinogen did not significantly differ from those in rats treated with BEP neuron transplants and vehicle but differed from those in rats treated with control transplants plus carcinogen (total prostate weight; mg/100 g body weight; BEP neurons+vehicle-213±13; BEP neurons+carcinogen-396±50; CONT+carcinogen-908±109; P<0.001, CONT+carcinogen vs. the rest; N=11-14). The prostates of rats receiving control cell transplants plus carcinogen displayed glandular hyperplasia (FIG. 11A), PIN (FIG. 11B) and occasionally well-differentiated adenocarcinoma with invasion (FIG. 11C). These lesions were primarily localized in the dorsolateral and anterior prostate. Similar to the non-carcinogen-treated controls, rats receiving BEP neuronal transplants and carcinogen showed either normal prostatic morphology (FIG. 11D) or mild epithelial atypia with glandular crowding (FIG. 11E). The incidence of adenocarcinoma was markedly lower (only 1 out of 17 rats examined; P<0.03) in the carcinogen plus BEP neuron transplant group than those rats receiving carcinogen and control cell transplants (22 out of 24 rats examined; Table 1).

TABLE 1
Effect of BEP cell transplants on prostatic neoplasia

| | | % | % | % |
|---|---|---|---|---|
| | % normal | atypia | hyperplasia | neoplasia |
| Treatment | (n) | (n) | (n) | (n) |
| BEP cells + Vehicle | 67 (14) | 33 (7) | 0 | 0 |
| CONT + Carcinogen | 3 (1) | 0 | 48 (11)^{a} | 48 (11)^{a} |
| BEP cells + Carcinogen | 65 (11) | 29 (5) | 6 (1) | 0 |

BEP cells or cortical cells (controls; CONT) were transplanted into the PVN of male rats. The rats were then treated with MNU and testosterone or with vehicle.
^{a}P < 0.03, % neoplasia and % hyperplasia in CONT vs. BEP cells as determined by Fisher's exact test.

BEP neuronal transplants increased NK cell cytolytic function and altered production of IFN-gamma Since NK cells with potent cytotoxic activity are known to effectively kill prostate cancer cells, we investigated whether BEP cell transplants altered the NK cell cytolytic function. The effect of BEP cells on inflammatory and anti-inflammatory cytokine levels in circulation was also studied, since this peptide is known to have potent anti-inflammatory effects in the body. Additionally, epidemiologic studies, together with laboratory and clinical studies, suggest that infection and inflammation contribute to the early development of prostate cancer. These issues were investigated by determining whether BEP cells can elevate NK cell function and alter circulatory levels of inflammatory and anti-inflammatory cytokines in rats. To characterize the influence of BEP cell transplants influence on NK cells, the effects of these transplants either in one PVN or in both PVN of rats were determined. Data showed that BEP neuron-induced activation of splenic NK cell cytolytic function was greater when these cells were transplanted in both PVNs as compared to only one PVN. BEP transplants produced similar dose-response effects on NK cell cytolytic activity in PBMC and on IFN-gamma levels in plasma of rats. In contrast, BEP transplants dose-dependently reduced levels of TNF-alpha levels in the plasma of rats. These results suggest that BEP cell transplants are effective in activating NK cell cytolytic functions and reducing the body's inflammatory milieu.

Discussion

The main finding was that under appropriate conditions, NSCs can be generated from rat embryonic hypothalamic tissues and propagated by cAMP-elevating agents to produce BEP neurons in cultures. Like in vivo, these cells go on to produce and secrete BEP, and they respond positively to the neuromodulator challenge. When transplanted in the hypothalamus, BEP cells survive and produce the peptide hormone. These BEP cell transplants inhibit prostate tumor development, possibly by increasing the NK cell activity, reducing the body's inflammatory milieu and by yet unknown immune surveillance mechanisms. These results identify a critical role for cAMP in the differentiation of BEP neurons and reveal a novel role of these neurons in controlling prostate tumor growth.

PACAP belongs to the peptide family that includes secretin, glucagons, growth hormone-releasing factor and vasoactive-intestinal peptide. Recent data suggest that PACAP affects developmental processes. PACAP gene expression and PACAP immunoreactivity are widely distributed in neurons within the neonatal rat brain. Activation of PACAP receptors regulates the proliferation of developing neuroblasts. PACAP and its receptors are expressed in the embryonic neural tube, where they appear to regulate neurogenesis. The activation of PACAP signaling in vitro has been shown to enhance NSC proliferation/survival through a PKA-independent mechanism. In contrast, PACAP has been shown to promote NSC self-renewal and neurogenesis through a mechanism dependent on PKA activation. Our results demonstrate that PACAP and dbcAMP, which is also an activator of PKA, interact to control the differentiation and functional maturation of BEP neurons.

We have previously shown that dbcAMP acts as a neurotropic factor for immature BEP neurons. Also, studies of transduction pathways identified the cAMP system as an important second messenger system in the regulation of hormone secretion and POMC gene regulation. Since PACAP is a potent inducer of cellular levels of cAMP in developing neurons, the interactive actions we observed between this peptide and dbcAMP support a role of the cAMP signaling system in the regulation of early differentiation of BEP neurons.

Chronic high levels of stress are known to enhance carcinogenesis in rat models (33). Furthermore, behavioral interventions aimed at reducing stress and increasing optimism in cancer patients have been documented to enhance immunity and to reduce tumor growth. These studies have identified the importance of stress regulation in the management of cancer growth. The data presented here show that the NSC-derived BEP neurons, when transplanted in the PVN, remain at the site of transplantation and are well integrated with the other cells at this site. They appear to be functional as significant amounts of POMC mRNA and BEP peptide were detected in the tissue containing the transplanted cells but not in the tissue containing control cells. The transplanted ex vivo produced BEP neurons also showed remarkable anti-tumor activity. These data suggest that stress-relieving BEP neurons have the ability to suppress the growth of prostate cancers.

In this study, we found that ex vivo-produced BEP neuronal transplants significantly increased NK cell cytolytic activity in the spleen and in the PBMC. In addition, the transplants increased IFN-gamma levels in plasma while reducing TNF-alpha levels. These data are in agreement with the finding that administration of BEP peptide in the PVN increases NK cell function. Because the transplants increased BEP levels in the hypothalamus but not in plasma, it is possible that the peptide may have inhibited sympathetic input to the spleen to increase NK cell cytolytic function. The NK cell is a critical component of the innate immune system and plays a central role in host defense against tumor cells. The importance of the NK cell in controlling tumor growth and metastasis of cancer cells has been clearly demonstrated in severe combined immunodeficiency mice. Hence, the possibility arises that the higher level of NK cell cytolytic activity may have caused unfavorable conditions for prostate cancer cell growth. In addition, in the BEP cell-treated animals the lower inflammatory milieu that was achieved by the higher level of anti-inflammatory IFN-gamma and lower level of inflammatory TNF-alpha may have also been involved in inhibiting prostate cancer growth. Proliferative inflammatory atrophy, a prostate cancer precursor lesion, ties the inflammatory response to prostatic carcinogenesis. Somatic epigenetic alterations, present in all prostate cancers, also appear to arise in the setting of inflammation. We hypothesize that BEP neuronal transplants inhibit prostate tumor development possibly by increasing the NK cell cytolytic activity and/or ameliorating the inflammation. These data provide strong evidence that hypothalamic BEP neurons play a critical role in controlling tumor growth. Because neuronal differentiation from NSC persists in the adult, the BEP-inducing therapies by cAMP-activating agents may hold promise as an adjuvant treatment for cancer.

Methods

Preparation of neurospheres from the fetal rat hypothalamus. Mediobasal hypothalamic tissues from fetal rats (embryonic day 17) of the Sprague Dawley (SD) strain (Charles River Laboratories, Wilmington, Mass.) were dissociated by mechanical dispersion as previously described. Neurons were separated from glial cells by filtering mixed hypothalamic cells through a 48-μm nylon mesh. Hypothalamic cells were then sedimented at 400 g for 10 min; pellets were resuspended in HEPES-buffered DMEM (HDMEM, 4.5 g/l glucose); and cells were cultured into 25-cm² polyornithine-coated tissue culture flasks (2.5 million cells/flask) in HDMEM containing 10% FBS and antibiotics (1% penicillin/streptomycin). On day 2, the culture medium was replaced with HDMEM containing 10% FBS, 33.6 μg/ml uridine, and 13.6 μg/ml 5-fluorodeoxyuridine to prevent the overgrowth of astroglial cells. On day 3, the culture medium was replaced with HDMEM containing serum supplement (SS; 30 nM selenium, 20 nM progesterone, 1 μM iron-free human transferrin, 5 μM insulin and 100 μM putrescin) and antibiotics. These chemicals were obtained from Sigma (St. Louis, Mo.), except FBS which was purchased from HyClone (Logan, Utah). Cells were maintained for the next 2 days in this medium. By this time, the cultures were approximately 85-90% neurons, as determined by MAP2 positivity.

Enriched hypothalamic neurons were maintained in HDMEM containing 10% FBS, trypsinized using trypsin/EDTA (Sigma) solution weekly and cultured for 3 wk to develop neurosphere. These spheres were then separated and dissociated into single cells by trypsinization and cultured in suspension or in poly-L-ornithine-coated 24-well plates (20,000 cells/well) in stem cell medium (DMEM F-12, lymphokine inhibitory factor (LIF), 0.1 μg/ml; L-glutamine, 10 mM; rat bFGF, 20 ng/ml; Mem amino acid solution, MAA, 0.5%; all of the chemicals were from Sigma except bFGF, which was obtained from R&D Systems, Minneapolis, Minn.) for a period of 2 wk, during which time they grew and developed secondary spheres. These neurospheres can be maintained in cultures for several months by regularly changing the medium and by splitting the cells. The secondary spheres were resuspended and cultured on poly-L-ornithine-coated 24-well plates (20,000 cells/well; for biochemical studies) or in poly-L-ornithine coated 8-well permanox slides (1,000 cells/slide; Nalg Nunc International Corp., IL; for histochemical studies). Differentiation experiments were performed by treating these cells for 1 wk with PACAP (1 or 10 μM; SynPep) and/or dbcAMP (1 or 10 μM; Sigma) and then suspending them in a defined cell culture medium without the drugs for 1 wk. At days 3, 7 and 14, the immunocytochemical, biochemical and/or qRT-PCR analyses were performed.

Immunohistochemical characterization of NSCs. Cell cultures were fixed in 4% paraformaldehyde for 30 min and then in 70% ethanol for an additional 30 min. Cells were incubated with primary antibodies overnight at 4° C. Primary antibodies used were monoclonal antibodies for nestin (BD Biosciences, San Jose, Calif.; 1 μg/ml), vimentin (clone V9, mouse ascites fluid, 0.22 μg/ml; Sigma; 1:40), a-internexin (Santa Cruz Biotechnology, Santa Cruz, Calif.; 1 μg/ml), MAP2 (2A+2B, clone AP-20, mouse ascites fluid, 0.72 μg/ml; Sigma), β-tubulin (clone SDL.3D10, 0.30 μg/ml, Sigma), GFAP (clone G-A-5, 45 μg/ml, Sigma), NF-M (145 kDa, 5 μg/ml, Chemicon International, Temecula, Calif.), RIP (anti-oligodendrocyte, clone NS-1, 1:000; Chemicon), TH (1:500; BD), polyclonal primary rabbit antibody for BEP (1:1000; Peninsula Laboratories, San Carlos, Calif.), GnRH (1:500; Chemicon) and NPY (1:500; Peninsula Laboratories). The secondary antibody used to react with mouse primary antibodies was Alexa Fluor 488 donkey anti-mouse IgG (4 μg/ml) and with the rabbit primary antibody was Alexa Fluor 594 donkey anti-rabbit IgG (H+L) (4 μg/ml; both from Molecular Probes, Eugene, Oreg.). Both of these secondary antibodies failed to stain cells in the absence of a primary antibody. Some cell cultures were mounted using DAPI-containing Mounting Medium (Vector Laboratories Inc. Burlingame, Calif.).

Expression levels of POMC mRNA in stem cells and differentiated cells were assayed by a quantitative RT-PCR (qRT-PCR) on an ABI PRISM 7700 Sequence Detector (Perkin Elmer Applied Biosystems, Foster City, Calif.), as described previously (38). The immunoreactive BEP levels in culture media were measured by a radioimmunoassay (RIA; 4).

Animal preparation and surgery. Pregnant female Sprague Dawley rats were obtained from Charles River Laboratories (Wilmington, Mass.) and housed in a controlled environment and provided rodent chow meal and water ad libitum. Male pups of these dams were used in these series of studies.

In vitro differentiated BEP cells were dissociated using 0.05% trypsin/EDTA, washed and resuspended in HDMEM and SS medium for transplantation. Cortical cells were prepared (39) and maintained in cultures for 4 days, trypsinized, and resuspended in HDMEM and SS medium for transplantation. Cells were placed on ice throughout the grafting session. Cell viability, assessed by the Trypan Blue exclusion assay, was routinely greater than 90%. The composition of the differentiated cultures, with respect to the absence of undifferentiated NSCs and the presence of mature BEP-producing cells, was verified before grafting by staining for nestin, MAP-2, GFAP, RIP (for oligodendrocytes) and BEP. In some experiments, BEP cells were frozen and thawed for 3 cycles and used as a non-viable BEP cells control.

Male rats between 35-60 days of age were anesthetized with sodium pentobarbital (50-70 mg/kg, i.p.; Henry Schein, Indianapolis, Ind.) and injected with 1.0 μl of cell suspension (20,000 cells/lobe) into either one or both PVN lobes (the coordinates were set 0.5 mm from the midline, 1.8 mm behind the bregma, 0.5 mm lateral of the bregma and 7.5 mm below the cortex) using a 5-μl Hamilton syringe. Each injection was over a 5-min duration. Following the injection, the cannula was left in place for 20 min to prevent cells from being sucked out during removal of the cannula. The cannula was then slowly removed over a 10-min period. The dura was closed with 9-0 suture, muscle was reapposed and the skin was closed with wound clips. Animal surgery and care were performed in accordance with institutional guidelines and complied with NIH policy.

NSC-BEP cell transplants viability. A group of male rats with differentiated BEP cell transplant in one of the PVN lobe was used for detection of BEP immunoreactivity in the transplanted cells. These animals were anesthetized with sodium pentobarbital and perfused with 0.1 M PBS followed by 4% paraformaldehyde in PBS, post-fixed, frozen in cryoprotectant, serially sectioned (40 μm) and double-stained for BrdU and BEP using immunohistochemistry methods. A separate group of rats with the differentiated BEP cell transplant or non-viable BEP cells transplant in one of the PVN lobe were used for measurement of POMC mRNA using the qRT-PCR analysis and BEP levels in the PVN and plasma using RIA.

NSC-BEP cell transplants and the growth of prostate tumors induced by MNU and testosterone in rats. The effects of the BEP cell transplants on MNU and testosterone-induced prostate tumor growth were determined as described previously. Adult male rats were transplanted with BEP cells or cortical cells into both PVN at 90 days of age and then were injected i.p. with cyproterone acetate (50 mg/0.3 ml DMSO/kg; Sigma) for 21 consecutive days followed by daily i.p. injections of 100 mg/kg testosterone propionate (Steraloids, Inc., Newport, R.I.) in propylene glycol for 3 days. One day after the last testosterone injection all rats received a single i.p. dose (50 mg/kg bw) of MNU (Sigma) dissolved in saline at 10 mg/ml. One week after MNU administration, rats received daily i.p. injection of testosterone (2 mg/kg bw) for 60 d. After this treatment period, rats were injected i.p. with 0.3 ml saline or LPS (100 μg/ml saline/kg), and 3 h later they were sacrificed, trunk blood was collected for the corticosterone ELISA assay (Diagnostic System Laboratories, Webster, Tex.). Prostates were removed from the adhering connective tissue, washed several times with physiological saline, weighed, fixed with 10% neutral buffered formalin and stained with hematoxylin and eosin for determination of tissue histopathology.

Innate immune system response to BEP cell transplants. The immune system response to BEP cell transplants was determined by measuring the NK cell cytolytic activity in the spleen and PBMC and cytokine (IFN-gamma and TNF-alpha) levels in the plasma following 4 wk of transplantation with BEP cells or cortical cells into one or both PVNs in male rats at 60-70 d of age. At the end of the experiment, these rats were decapitated and the spleens and peripheral blood were obtained and used for isolation of splenocytes and PBMC to measure NK cell cytolytic activity as described previously (6). Plasma levels of INF-gamma and TNF-alpha were measured by ELISA (Amersham Biosciences, Piscataway, N.J.).

Statistics. Means±standard errors of the data calculated and presented in the text. The significance of the differences between two experimental groups was analyzed by the t test. Multiple groups of data were analyzed using one-way analysis of variance. The differences between groups were determined using the Student-Newmann-Keuls test. The proportions of tumors of each type developed between treatments groups were compared using the Fisher's exact test. A value of P<0.05 was considered significant.

### Example 3

### Beta-Endorphin Neuron Transplants in the Hypothalamus Reduces Rheumatoid Arthritis Development in a Rat Model

It was shown recently that implantation of stem cell-derived beta-endorphin producing (BEP) neurons into the rat hypothalamus is capable in blocking components of inflammation, including proinflammatory cytokine production in a cancer model. In this study, we are reporting the effect of the BEP cells transplantation into the paraventricular nucleus of the hypothalamus on the systemic inflammation exemplified by adjuvant-induced arthritis (AIA), which is a preclinical in vivo model of rheumatoid arthritis. In this model, young female Lewis rats were transplanted with BEP cells or the neuronal cortical cells into the paraventricular nuclei. After the end of the recovery period, AIA was induced by intradermal injection of the oil suspension of Mycobacterium butyricum at the base of the tail. After the onset of the disease, the severity of the AIA in the animals was assessed by measuring ankle circumference, paw thickness and scoring the extent of paw edema (artcular index). The animals were monitored for three weeks after the injection. There were statistically significant reductions in the articular index (P<0.05 at day 12, P<0.02 at day 21; n=6-8) and posterior paw thickness (P<0.05 at day 16, P<0.04 at day 21; n=7-8) in BEP neuron transplanted rats but not in control neuron transplanted rats. Ankle circumferences were also decreased in the BEP-treated cells in comparison to control (P<0.01 at day 21; n=7-8). This study demonstrates for the first time that activation of BEP cell in the rat hypothalamus may protect from rheumatoid arthritis development. (Supported by NIH Grant AA 08757).

### Example 4

Neural stem cell-derived beta-endorphin neuron transplants into the brain increase natural killer cell activity, decrease antiinflammatory cytokines and prevent metastatic colonization in rats.

A review of studies that evaluated psychological factors and outcome in cancer patients suggested an association between certain psychological factors and the growth and metastasis of cancer. It has been suggested that the effects of stress on the immune system may in turn affect the growth of some tumors. We have previously shown that a set of hormone secreting nerve cells in the hypothalamus, called beta-endorphin producing (BEP) neurons, plays a role in regulating both the stress response and immune function. Hence, we tested whether activation of BEP neurons may help inhibit metastatic colonization. To test this we differentiated rat neural stem cells from the hypothalamus into BEP neurons with the aid of cAMP-activating agents in culture, which were later transplanted into the paraventricular nuclei (PVN) of the hypothalamus of live Fischer 344 rats. Control rats were transplanted with cortical cells or not operated. Following 3 weeks after cell transplantation, these rats were inoculated intravenously with rat mammary tumor cells (MADB106 tumor cells) for the assessment of lung tumor retention (LTR). Additionally, the impact of cell transplants on plasma levels of stress hormone corticosterone and cytokines interferon gamma (INF-gamma, tumor necrosis factor alpha (TNF-alpha and interleukin 6 (IL6) in plasma and the levels of natural killer (NK) cell cytotoxicity in peripheral blood and spleen was evaluated. When the in vitro differentiated BEP neurons were transplanted into the PVN of an adult rat, they integrated well with the surrounding cells and produced BEP and it precursor gene proopiomelanocortin. The animal with BEP neuron transplants showed no retention of MADB106 cells in lung or other tissues, whereas the animals with cortical cell transplants or no transplants showed significant retention of these cells and visible surface metastasis in the lungs. Rats with BEP neuron transplants also showed increased NK cell cytolytic function in the spleens and peripheral blood mononuclear cells, elevated levels of anti-inflammatory cytokine INF-gamma and decreased levels of inflammatory cytokine TNF-alpha in plasma. These results identify a protective role of the BEP neuron against the metastatic diffusion possibly via increasing the innate immune function and reducing the inflammatory milieu. (Supported by NIH Grant AA015718).

### Example 5

### Prenatal Alcohol Exposures Reduces Progenitor Cells Differentiation to BEP Neurons while Nanosphere's Delivered Camp Increases BEP Neuron Growth

We have recently demonstrated that prenatal exposure to ethanol causes a significant decrease in the number of beta-endorphin (BEP) neurons in the hypothalamus in rats. Although ethanol is known to induce BEP neuronal apoptotic death, it is not known whether it also alters differentiation of BEP neurons from neuronal progenitor cells. Using an early postnatal binge ethanol administration model and bromdeoxyuridine (BrdU) labeling method, we show here that ethanol reduced the number of differentiated BEP neurons. Similarly, moderate dose of ethanol inhibited the differentiation and maturation of BEP neurons by cAMP-activating agents in primary cultures of fetal hypothalamic neuronal progenitor cells. Since the cAMP-activating agent increased BEP neuron differentiation in vitro, we tested the effect of nanosphere-delivered cAMP into the 3rd ventricle on endogenous progenitor cell differentiation to BEP neurons in adult male rats. We found, nanosphere-delivered cAMP significantly increased the number of BEP neurons in these rats. Functional studies using NK cell response to lipopolysaccaride in the nanosphere-treated rats verified the histological data of BEP neuronal growth. These data suggest that prenatal ethanol exposure alters progenitor cell differentiation to reduce the number of BEP cells. Furthermore, the data provide first evidence to show the potential of nanosphere's delivered cAMP to generate new BEP neurons to reduce immune and stress problems in fetal alcohol exposed subjects. (Supported by NIH Grant AA08757)

The disclosures of each reference provided herein are hereby incorporated by reference in their entireties.

### REFERENCES

Ahmed M S, Llanos J, Blatties C M 1984 Interleukin-1 interacts with opioid binding sites. Proc Soc Neurosci Abstr 10:1109.
Altman J, Bayer S A (1978) Development of the diencephalon in the rat. III. Ontogeny of the specialized ventricular linings of the hypothalamic third ventricle. J. Comp. Neurol. 182:995-1015
Arai S, Kingemann H G (2005) Natural killer cells: can they be useful as adoptive immunotherapy for cancer? Expert Opin Biol Ther. 5:163-172.
Arjona A, Boyadjieva N, Kuhn P, Sarkar D K. (2006) Fetal ethanol exposure disrupts the daily rhythms of splenic granzyme B, IFN-gamma, and NK cell cytotoxicity in adulthood. Alcohol Clin Exp Res. 30:1039-1044.
Arjona A, Boyadjieva N, Sarkar D K. (2004) Circadian Rhythms of granzyme B, perforin, IFN-γ and natural killer cell cytolytic activity in the spleen: effects of chronic ethanol. Journal of Immunology 172:2811-2817.
Arunkumar A, Vijayababu M R, Venkataraman P, Senthilkumar K, Arunakaran J. (2006) Chemoprevention of rat prostate carcinogenesis by diallyl disulfide, an organosulfur compound of garlic. Biol Pharm Bull. 29:375-379.
Austin Taylor M, Bennett M, Kumar V, Schatzle J D. (2000) Functional defects of NK cells treated with chloroquine mimic the lytic defects observed in perforin-deficient mice. J Immunol 165:5048-5053.
Barry M, Bleackley R C. (2002) Cytotoxic T lymphocytes: all roads lead to death. Nat Rev Immunol 2:401-409.
Bernstein H G, Krell D, Emrich H M, Baumann B, Danos P, Diekmann S, Bogerts B (2002) Fewer β-endorphin expressing arcuate nucleus neurons and reduced beta-endorphinergic innervation of paraventricular neurons in schizophrenics and patients with depression. Cell. Mol. Biol. 48 Online Pub:OL259-65
Boyadjieva N Iv, Reddy R H R, De A and Sarkar D. K. (1998) Evidence for a role of nitric oxide in ethanol and interleukin-1β regulated β-endorphin release and proopiomelanocortin gene expression in hypothalamic neurons in primary cultures. Alcoholism Clinical & Experimental Research (supplement) 22:139
Boyadjieva N, Advis J P, Sarkar D K. (2006) Role of BEP, corticotropin-releasing hormone and autonomic nervous system in mediation of the effect of chronic ethanol on natural killer cell cytolytic activity. Alcohol Clin Exp Res 30:1761-1767.
Boyadjieva N, Meadows G G, Sarkar D K (2001) Chronic ethanol inhibits natural killer cell cytolytic activity: role of opioid peptide β-endorphin. J. Immunol. 167:5645-5652.
Boyadjieva N, Meadows G G, Sarkar D K. (2001) Chronic ethanol inhibits natural killer cell cytolytic activity: role of opioid peptide beta-endorphin. Journal of Immunology 167:5645-5652.
Boyadjieva N, Sarkar D K (1997) Effects of ethanol on basal and prostaglandin E1-induced increases in beta-endorphin release and intracellular cAMP levels in hypothalamic cells. Alcohol Clin Exp Res 21:1005-1009
Boyadjieva N I, Chaturvedi K, Poplawski M M, Sarkar D K (2004) Opioid antagonist naltrexone disrupts feedback interaction between μ- and δ-opioid receptors in splenocytes to prevent alcohol inhibition of NK cell function. Journal of Immunology 173:42-49.
Boyadjieva N I, Dokur M, Advis J P, Meadows G G, Sarkar D K (2002) Beta-endorphin modulation of lymphocyte proliferation: effects of ethanol. Alcoholism: Clinical and Experimental Research 26:1719-27.
Boyadjieva, N., De, A., Fung, S. J. and Sarkar, D. K. (1995) Interleukin-1β inhibits in vivo and in vitro β-endorphin secretion from hypothalamic neurons by activating nitic oxide synthase. Ann. Meet. Soc. Neurosciences, Abstr. 814.15.
Carden M J, Trojanowski J Q, Schlaepfer W W, Lee V M (1987) Two-stage expression of neurofilament polypeptides during rat neurogenesis with early establishment of adult phosphorylation patterns. J Neurosci. 7:3489-3504
Castro M G, Morrison E (1997) Post-translational processing of proopiomelanocortin in the pituitary and in the brain. Crit. Rev. Neurobiol. 11:35-57
Chaudhuri S, Law S (2005) Stem cells and frontiers of therapeutic advances in cancer. J Exp Clin Cancer Res. 24:165-173.
Chen C P, Boyadjieva N, Advis J P, Sarkar D K. (2006) Ethanol suppression of the hypothalamic POMC level and the splenic NK cell cytolytic activity is associated with a reduction in the expression of proinflammatory cytokines but not antiinflammatory cytokines in neuroendocrine and immune cells. Alcohol Clin Exp Res 30:1925-1932.
Chen C P, Kuhn P, Advis J P, Sarkar D K (2004) Chronic ethanol consumption impairs the circadian rhythm of proopiomelanocortin and period genes mRNA expression in the hypothalamus of the male rat. J. Neurochem. 88:1547-1554
Colucci F, Caligiuri M A, Di Santo J P. (2003) What does it take to make a natural killer? Nat Rev Immunol 3:413-425.
Cone R D, Cowley M A, Butler A A, Fan W, Marks D L, Low M J (2001) The arcuate nucleus as a conduit for diverse signals relevant to energy homeostasis. Int. J. Obes. Relat. Metab. Disord. 25 Suppl 5:S63-67
Dafny N. 1983 Modification of morphine withdrawal by interferon. Life Sci 32; 303-305.
De A, Boyadjieva N, Pastorcic M, Reddy B V, Sarkar D K (1994) cAMP and ethanol interact to control apoptosis and differentiation in hypothalamic beta-endorphin neurons. J. Biol. Chem. 269:26697-26705
Dewan M Z, Terunuma H, Ahmed S, Ohba K, Takada M, Tanaka Y, Toi M, Yamamoto N. (2005) Natural killer cells in breast cancer cell growth and metastasis in SCID mice. Biomed Pharmacother. 59 Suppl 2:S375-379.
Dewan M Z, Terunuma H, Takada M, Tanaka Y, Abe H, Sata T, Toi M, Yamamoto N (2006) Role of natural killer cells in hormone-independent rapid tumor formation and spontaneous metastasis of breast cancer cells in vivo. Breast Cancer Res Treat. (2006) Oct. 26; [Epub ahead of print]
DiCicco-Bloom E, Deutsch P J, Maltzman J, Zhang J, Pintar J E, Zheng J, Friedman W F, Zhou X, Zaremba T (2000) A expression and ontogenetic functions of the PACAP ligand/receptor system during sympathetic development. Dev Biol 219:197-213
Dokur M, Boyadjieva N, Sarkar D K (2003) Reduction of perforin, granzyme B and cytokine IFN-gamma by ethanol in male Fischer-344 rats. Alcoholism: Clinical and Experimental Research 27:670-676.
Dokur M, Boyadjieva N, Sarkar D K (2004) Catecholaminergic control of NK cell cytolytic activity regulatory factors in the spleen. Journal of Neuroimmunology 151:148-157.
Dokur M, Boyadjieva N I, Advis J P, Sarkar D K (2004) Modulation of hypothalamic β-endorphin-regulated expression of natural killer cell cytolytic activity regulatory factors by ethanol in male Fisher-344 rats. Alcoholism: Clinical and Experimental Research 28:1180-1186.
Dokur M, Chen C P, Advis, J P, Sarkar D K (2005) β-Endorphin Modulation of Interferon-γ, Perforin and Granzyme B Levels in Splenic NK cells: Effects of ethanol. J Neuroimmunology 166:29-3
Evans J, Sumners C, Moore J, Huentelman M J, Deng J, Gelband C H, Shaw G (2002) Characterization of mitotic neurons derived from adult rat hypothalamus and brain stem. J Neurophysiol. 87:1076-1085
Faith R E, Liang H J, Murgo A J and Plotnikoff N P (1984) Neuroimmunomodulation with enkephalins:enhancement of human natural killer (NK) cell activity in vitro. Clin Immunol Immunopathol 31:412-418.
Hansel D E, Eipper B A, Ronnett G V (2001) Regulation of olfactory neurogenesis by amidated neuropeptides. J Neurosci Res. 66:1-7
Irwin M, Hauger R L, Brown M, Britton K T (1988) CRF activates autonomic nervous system and reduces natural killer cytotoxicity. Am J Physiol 255:R744-747.
Ishikawa E, Tsuboi K, Saijo K, Harada H, Takano S, Nose T, Ohno T (2004) Autologous natural killer cell therapy for human recurrent malignant glioma. Anticancer Res. 24:1861-1871.
Janeway C A Jr, Medzhitov R. (2002) Innate immune recognition. Arum Rev Immunol. 20:197-216.
Kaplan M P, Chin S S, Fliegner K H, Liem R K (1990) Alpha-internexin, a novel neuronal intermediate filament protein, precedes the low molecular weight neurofilament protein (NF-L) in the developing rat brain. J Neurosci. 10:2735-2748
Kay N E, Allen J, Morley J E 1984 Endorphins stimulate normal human peripheral blood lymphocyte natural killer activity. Life Sci 35:53-59.
Lee M, Lelievre V, Zhao P, Torres M, Rodriguez W, Byun J Y, Doshi S, Ioffe Y, Gupta G, de los Monteros A E, de Vellis J, Waschek J (2001) Pituitary Pituitary adenylyl cyclase-activating polypeptide stimulates DNA synthesis but delays maturation of oligodendrocyte progenitors. J Neurosci. 21:3849-3859
Lee S, Schmidt D, Tilders F, Rivier C (2000) Increased activity of the hypothalamic-pituitaru-adrenal axis of rats exposed to alcohol in utero: role of altered pituitary and hypothalamic functiion. Mol. Cell. Neurosci. 16:515-528.
Lendahl U, Zimmerman L B, McKay R D (1990) CNS stem cells express a new class of intermediate filament protein. Cell 60:585-595
Li S, Grinevich V, Fournier A, Pelletier G (1996) Effects of pituitary adenylate cyclase-activating polypeptide (PACAP) on gonadotropin-releasing hormone and somatostatin gene expression in the rat brain. Brain Res Mol Brain Res. 41:157-62
Lundblad J R, Roberts J L (1988) Regulation of proopiomelanocortin gene expression in pituitary. Endocr. Rev. 9:135-158
Markakis E A, Swanson L W (1997) Spatiotemporal patterns of secretomotor neuron generation in the parvicellular neuroendocrine system. Brain Res. Rev. 24:255-291
Mathews P M, Froelich C J, Sibbit W L Jr and Bankhurst A D (1983) Enhancement of natural cytotoxicity by β-endorphin J Immunol 130:1658-1662.
Miller J S. (2001) The biology of natural killer cells in cancer, infection, and pregnancy. Exp Hematol 29:1157-1168.
Mizuno Y, Kondo K, Terashima Y, Arima H, Murase T, Oiso Y (1998) Anorectic effect of pituitary adenylate cyclase activating polypeptide (PACAP) in rats: lack of evidence for involvement of hypothalamic neuropeptide gene expression. J Neuroendocrinol.10:611-616
Moretta L, Bottino C, Pende D, Mingari M C, Biassoni R, Moretta A. (2002) Human natural killer cells: their origin, receptors and function. Eur J Immunol 32:1205-1211.
Nielsen H S, Hannibal J, Fahrenkrug J (1998) Embryonic expression of pituitary adenylate cyclase-activating polypeptide in sensory and autonomic ganglia and in spinal cord of the rat. J Comp Neurol 394:403-415
Nielsen H S, Hannibal J, Fahrenkrug J (1998) Expression of pituitary adenylate cyclase activating polypeptide (PACAP) in the postnatal and adult rat cerebellar cortex. Neuroreport. 9:2639-2642
Olcese J, McArdle C A, Middendorff R, Greenland K (1997) Pituitary adenylate cyclase-activating peptide and vasoactive intestinal peptide receptor expression in immortalized LHRH neurons. J Neuroendocrinol. 9:937-943
Otraldo J R, Gerard J P, Henderson L E, Neubauer R H and Rabin H (1983) Responsiveness of purified natural keller cells to pure interleukin-2 (IL-2). In: Interleukin Lymphokines and Cytokines (eds) Oppenheim J J and Rabin H, p 63, Academic Press, New York.
Pankov IuA, Iatsyshina S B, Karpova S K, Chekhranova M K, Popova IuP, Grigorian O N, Rogaev E I (2002) Screening of mutations in genes of proopiomelanocortin in patients with constitutional exogenous obesity. Vopr Med Khim. 48:121-30
Perussia B, Starr S, Abraham S, Fanning V, Trinchieri G (1983) Human natural killer cells analyzed by B73.1, a monoclonal antibody blocking Fc receptor functions. I. Characterization of the lymphocyte subset reactive with B73.1. J Immunol 130:2133-2141
Plotsky P M (1991) Pathways to the secretion of adrenocorticotropin: A review from the portal. J Endocrinol 3:1.
Pross H F, Baines M G, Rubin P, Shragge P, Patterson M S (1981) Spontaneous human lymphocyte-mediated cytotoxicity against tumor target cells. IX. The quantitation of natural killer cell activity. J Clin Immunol 1:51-63.
Raja S M, Wang B, Dantuluri M, Desai U R, Demeler B, Spiegel K, Metkar S S, Froelich C J (2002) Cytotoxic cell granule-mediated apoptosis. Characterization of the macromolecular complex of granzyme B with serglycin. J Biol Chem 277:49523-49530
Raju T, Bignami A, Dahl D (1981) In vivo and in vitro differentiation of neurons and astrocytes in the rat embryo. Immunofluorescence study with neurofilament and glial filament antisera. Dev. Biol. 85:344-357
Reglodi D, Lubics A, Tamas A, Szalontay L, Lengvari I (2004) Pituitary adenylate cyclase activating polypeptide protects dopaminergic neurons and improves behavioral deficits in a rat model of Parkinson's disease. Behav Brain Res. 151:303-312
Roisen F J, Klueber K M, Lu C L, Hatcher L M, Dozier A, Shields C B, Maguire S (2001) Adult human olfactory stem cells. Brain Res. 890:11-22
Roybon L, Christophersen N S, Brundin P, Li J Y (2004) Stem cell therapy for Parkinson's disease: where do we stand? Cell Tissue Res. 318:261-273
Sarkar D K, Kuhn P, Marano J, Chen C, Boyadjieva N (2007) Alcohol exposure during the developmental period induces beta-rndorphin neuronal death and causes alteration in the opioid control of stress axis function. Endocrinology 148:2828-2834.
Santoli D and Koprowski N (1979) Mechanisms of activation of human natural killer cells against tumor and virus-infected cells. Immunol Rev 44:125.
Settle M (2000) The hypothalamus. Neonatal Netw. 19:9-14
Shaw G (1998) Neurofilaments. Springer-Verlag, Berlin.
Sherwood N M, Krueckl S L, McRory J E (2000) The origin and function of the pituitary adenylate cyclase-activating polypeptide (PACAP)/glucagon superfamily. Endocr. Rev. 21:619-670
Smith M J, Hayakawa Y, Takeda K, Yagta H (2002) New aspects of natural killer cell survellance and therapy of cancer. Nature Rev Cancer 2:850-861.
Smith M J, Jennes L (2001) Neural signals that regulate GnRH neurons directly during the oestrous cycle. Reproduction 122:1-10
Taylor A N, Branch B J, Van Zuylen J E, Redei E (1988) Maternal alcoholism and stress responsiveness in offspring. Adv. Exp. Med. Biol. 245: 311-317.
Terenius L (2000) From opiate pharmacology to opioid peptide physiology. Ups. J. Med. Sci. 105:1-15
Trinchieri G (1989) Biology of natural killer cells. Adv Immunol 47:187-376.
Van den Berghe G (2000) Novel insights into the neuroendocrinology of critical illness. Euro J. Endocrinol. 143:1-13
van Eerdenburg F J, Rakic P (1994) Early neurogenesis in the anterior hypothalamus of the rhesus monkey. Brain. Res. Dev. Brain. Res. 79:290-296
Vescovi A L, Reynolds B A, Fraser D D, Weiss S (1993) bFGF regulates the proliferative fate of unipotent (neuronal) and bipotent (neuronal/astroglial) EGF-generated CNS progenitor cells. Neuron 11:951-966
Yang Z, Huang W, Lee D, Copolov D L, Lim A T (1993) The adenylyl cyclase-cyclic AMP system modulates morphological and functional development of hypothalamic beta-endorphin neurons in culture. J. Neuroendocrinol. 5:371-380
Zangen A, Nakash R, Roth-Deri I, Overstreet D H, Yadid G (2002) Impaired release of beta-endorphin in response to serotonin in a rat model of depression. Neuroscience 110:389-393
Ah D V, Kang D H, Carpenter J S (2007) Stress, optimism, and social support: impact on immune responses in breast cancer. Res Nurs Health 30:72-83.
Carlson L E, Speca M, Patel K D, Faris P (2007) One year pre-post intervention follow-up of psychological, immune, endocrine and blood pressure outcomes of mindfulness-based stress reduction (MBSR) in breast and prostate cancer outpatients. Brain Behav Immun 21:1038-49.
Giovannucci E, Michaud D (2007) The role of obesity and related metabolic disturbances in cancers of the colon, prostate, and pancreas. Gastroenterology 132:2208-2225.
Grinshpoon A, Barchana M, Ponizovsky A, Lipshitz I, Nahon D, Tal O, Weizman A, Levav I (2005) Cancer in schizophrenia: Is the risk higher or lower? Schizophr Res 73:333-341.
Hisano S, Kawano H, Nishiyama T, Diakoku S (1982) Immunoreactive ACTH/beta endorphin neurons in the tubero-infundibular hypothalamus of rats. Cell Tissue Res 224:303-314.
Lagace D C, Whitman M C, Noonan M A, Ables J L, DeCarolis N A, Arguello A A, Donovan M H, Fischer S J, Farnbauch L A, Beech R D, DiLeone R J, Greer C A, Mandyam C D, Eisch A J (2007) Dynamic contribution of nestin-expressing stem cells to adult neurogenesis. J Neurosci 27:12623-12629.
Lissoni P, Barni S, Paolorossi F, Crispino S, Rovelli F, Ferri L, Delitala G, Tancini G. (1983) Evidence for altered opioid activity in patients with cancer. Br J Cancer 56:834-837.
Nelson W G (2007) Prostate cancer prevention. Curr Opin Urol 17:157-67.
Noh J S, Gwag B J (1997) Attenuation of oxidative neuronal necrosis by a dopamine D1 agonist in mouse cortical cell cultures. Exp Neurol 146:604-608.
Ohta S, Gregg C, Weiss S. (2006) Pituitary adenylate cyclase-activating polypeptide regulates forebrain neural stem cells and neurogenesis in vitro and in vivo. J Neurosci Res 84:1177-1186.
Spiegel D, Sephton S E (2001) Psychoneuroimmune and endocrine pathways in cancer: effects of stress and support. Semin Clin Neuropsychiatry 6:252-265.
Stagg J, Smyth M J. (2007) NK cell-based cancer immunotherapy. Drug News Perspect 20:155-163.50
Stanojević S, Mitić K, Vujić V, Kovacević-Jovanović V, Dimitrijević M (2006) Beta endorphin differentially affects inflammation in two inbred rat strains. Eur J Pharmacol 549:157-165.
Sutcliffe S, Platz E A (2007) Inflammation in the etiology of prostate cancer: an epidemiologic perspective. Urol Oncol 25:242-249.

1. A method of isolating neural stem cells from a fetal hypothalamus comprising: isolating mixed neural and neural stem cells from glial cells in a fetal hypothalamus, and growing the isolated mixed neural and neural stem cells for several generations in cultures so that only the neural stem cells remain in the cultures.

2. The method of claim 1, further comprising introducing an agent that kills the glial cells but not the neural and neural stem cells in cultures.

3. The method of claim 2, wherein the agent is selected from the group consisting of uridine, fluodeoxyuridine and a combination thereof.

4. The method of claim 1, further comprising maintaining the neural stem cells in cultures in the presence of stem cell medium with lymphokine inhibiting factor (LIF) so that only neural stem cells with the ability to differentiate into beta-endorphin (BEP) neurons remain in cultures.

5. The method of claim 4 wherein the cultures also contain basic fibroblast growth factor (bFGF).

6. The method of claim 4, further comprising differentiating beta-endorphin neuronal cells from neural stem cells that are under the influence of LIF by (i) removing the influence of LIF from the neural stem cells, (ii) maintaining the neural stem cells in an environment favoring the survival of neurons, and then (iii) treating the neural stem cells with a differentiating agent selected from group consisting of (a) pituitary adenylate cyclase activating peptide (PACAP), (b) dibutyryl cyclic adenylate cyclase (dbcAMP) and (c) a combination thereof.

7. A method of differentiating beta-endorphin neuronal cells from neural stem cells that are under the influence of LIF comprising: (i) removing the influence of LIF from the neural stem cells, (ii) maintaining the neural stem cells in an environment favoring the survival of neurons, and then (iii) treating the neural stem cells with a differentiating agent selected from group consisting of (a) pituitary adenylate cyclase activating peptide (PACAP), (b) dibutyryl cyclic adenylate cyclase (dbcAMP) and (c) a combination thereof.

8. The method of claim 6, wherein the environment in step (ii) is neuron culture media.

9. The method of claim 6, wherein the treating in step (iii) is performed for about 7 days.

10. The method of claim 6, wherein the agent in step (iii) is a combination of PACAP and dbcAMP.

11. A method of differentiating endogenous neural stem cells into BEP cells in a patient in need thereof comprising: (i) administering an effective amount of an agent selected from the group consisting of (a) pituitary adenylate cyclase activating peptide (PACAP), (b) dibutyryl cyclic adenylate cyclase (dbcAMP) and (c) a combination thereof into the central nervous system.

12. The method of claim 11, wherein the agent is administered into the brain.

13. The method of claim 12, wherein the agent is administered into the hypothalamus.

14. The method of claim 12, wherein the agent is administered into the third ventral.

15. The method of claim 11, wherein the agent is administered as a pharmaceutically acceptable nanosphere.

16. The method of claim 11, wherein the amount of agent administered is sufficient to provide BEP cell differentiation to reduce physiological stress responses.

17. The method of claim 11, wherein the amount of agent administered is sufficient to provide BEP differentiation to activate natural killer (NK) cells and inhibit proinflammatory cytokines.

18. The method of claim 11, wherein the amount of agent administered is sufficient to improve the innate immune response critical for defense against diseases selected from the group consisting of infectious diseases including viral and bacterial diseases, and hyperproliferative diseases such as cancers.

19. The method of claim 18, wherein the disease is neoplasia.

20. The method of claim 18, wherein the disease is cancer.

21. The method of claim 20, wherein the disease is metastatic breast cancer.

22. The method of claim 1, wherein the amount of agent administered is sufficient to provide BEP cell differentiation to reduce inflammation associated with immunologic diseases.

23. The method of claim 22, wherein the immunological disease is selected from the group consisting of rheumatoid arthritis, adult onset diabetes, thyroid disorder, celiac disease, inflammatory bowel syndrome, and a combination thereof.

24. The method of claim 7, wherein the environment in step (ii) is neuron culture media.

25. The method of claim 7, wherein the treating in step (iii) is performed for about 7 days.

26. The method of claim 7, wherein the agent in step (iii) is a combination of PACAP and dbcAMP.

27. The method of claim 20, wherein said cancer is prostate cancer or breast cancer.
